SEQUENCE LISTING (5 sequences)

SEQ ID NO: 1 - DNA, 4637 bp, Artificial
  Prolidase expression construct for nuclear transformation of Dunaliella salina
ttgacataaattgagtcctcaaggttaacaacctttaataaaagatgaatgcatagtctattttccattgaaaaatggttagtcataaatctctatgggattatgcggtttaatttgatgcaacgcaaagaaccttaccagggtttgacatgtctagaatttttcagaaatggaaaagtgcttcctttttatgaaaggaagaactagaacacaggtggtgcatggctgtcgtcagctcgtgcgttgacgtgtatggttaagtcctgcaacgagcgcaaccctcgtctttagttactttccaagttctctaaagagactgcgcgcgcaagcgctgaggaaggtgaggatgacgtcaagtcagcatgccccttacaccctgggcgacacgcgtaatacaatggttgggacaatcagaagctaccccgtaagggcacgccaatctgctaaactcaaccctagttcggattgtaggctgcaactcgcctacatgaagccggaatctatagtaatcgccagtcagctatatggcggtgaatacgttcctggtctctgtacacaccgcccgtcacatcaagaaaggtggtagtggattaagctactagtctaacctctctgaggaagacggtatccactccaagactactgatcatgatgaagtcgtaacaaggtaggggtactggaaggtgtccctggatcacctccttctttttaggaattttctatctgaccctcctaccaggagactaggacctttggtcctagcctcacggtaaacccaaagggtttaggcttaccttctcgtgaggggagctgacgcgttagatggaaaaataaaagtttttccatctataaagcgtaatgagatagaatcaataaaattatttaaaaggtgaccttaaccgaacgtaaattttacgttcggttaagtcacccttatatattcttatccaaaagaattttaatgggctattagctcagttggttagagcgcacccctgataagggtgaggtcagaagttcaaatcttctatagcccaccggacaaaacaactttagttaatatcgaagttttcaatatcctttaggatattgaaaatcttatagatttctataagaaaattctaatagagaagaaaatttacgaagtaaattttctattttcttcgataaattctgaaagagaagaaaatttactttgtaaatgttctattttcttccgaaaatttttttcgcggaaaaaacaaaagaaaaattttttttacgaaaaaaattttcccggacaaaactttgttttgtcctaaggaaaaaacaacaaagaaaattctgaaagaattttctttgttgttttgtccggcataaaaaaattttctttgtggttttgtcctaggaaaacccgtccccaaggcgggtccgttcgtccgtcgcgtgaggaggggggcacctttggttttttccgctgatggtttgtgtacgaggtcagcttccatgggcggggcggctgtttgcgatgctcttacgggcttaacacatgtatcttgtatagacctatttgtttatatcatttatcttaaaagaacaaaaaaaaaatttttggaggatcgtctactatggaaaaaaaaataacaggatatactacagtagacatttcacaatggcatcgtaaagaacatttcgaagcttttcaaagtgttgcacaatgtacttataaccaaacagttcaattagatattacagctttcttaaaaactgttaaaaaaaataaacacaaattttatccagcttttattcatattttagctcgtttaatgaatgctcatccagaatttcgtatggctatgaaagacggtgaattagttatttgggattcagttcatccttgttacactgtttttcacgaacaaacagaaactttctcatcattatggtcagaataccatgatgattttagacaatttttacacatttattcacaagatgtagcatgttatggtgaaaatttagcatattttcctaaaggttttattgaaaatatgttcttcgtttctgctaatccttgggtttctttcacttcttttgatttaaatgtagctaatatggataatttctttgcacctgtatttacaatgggtaaatattatacacaaggtgataaagttttaatgccattagcaattcaagtacatcatgctgtatgtgatggtttccacgtaggtcgtatgttaaacgaattacaacaatattgtgacgaatggcaaggtggtgctggaggatcgtctactatgcgtttagaaaaatttattcatttattaggtgaacgtggttttgatggtgcattaatttctcctggtacaaatttatattatttaactggtttaagattacatgaagtaggtgaacgtttagcaattttagcagtatcagctgaaggtgactatcgttttttagctccatctttatatgaaaacgtagtaaataattttccagctacattttggcatgatggtgaaaacccatacgcaaaattacgtgaaattttagaagaattaggtattagtaaaggaagaattttaattgaagacactatgcgtgctgattggttaattggtattatgaaattaggtaaatttacatttcaaccattaagttcattaattaaagaattacgtatgattaaagataaagaagaagttaaaatgatggaacatgctagtcgtattgcagataaagttttcgaagaaattttaacttgggatttaattggtatgaaagaacgtgaattagctttaaaaattgaattattaattcgtgaattatctgatggtattgcttttgaaccaattgttgcatctggagaaaatgctgctaatcctcatcatgaaccaggtgaacgtaaaattagaaaaggtgatattattattttagattatggtgctcgttggaaaggttattgttctgatattacaagaactattggtttaggagaattagatgaacgtttagtaaaaatttacgaagttgtaaaagacgctcaagaatcagcattcaaagctgtacgtgaaggtattaaagctaaagacgtagattctcgtgctcgtgaagtaatttctaaagcaggttatggtgaatacttcattcatcgtactggacatggtttaggtttagatgttcatgaagaaccttatattggtcctgatggagaagtaattttaaaaaatggtatgacatttactattgaaccaggtatttatgttccaggtttaggtggtgttcgtattgaagatgatattgtagtagatgaaggtaaaggtcgtcgtttaactaaagcagaaagagaattaattattttacaccaccaccaccaccactaattctaaatacttaagaattaagtagacttgtctacttaattctttttaatagcatgggtggagtaaactccacccatgctattatttctaacaactataaaataagatttttttatnctaaatatcgaagttttccatatcgcagaagtatcacttctgcgagatctccgcgtttttctatagaaaaacgcggacatagaaaatcttcgagaaatgaatttttgtaatagaatctaatttaattacacaaatgtaaattacaaaaaggattgagcttcaggttcagctatataacctggttctgctatataaacaatataacttgaaatatcagtagatgaacaatttaatttatattaatagtcgatgtccttcaataacgaatcgtatttttatacggtatgttgatatttatatattctcttatatgttatgaatatatatcatatgttacgataacccccctctggggttattgtgagatctcgtttctaaaaaaacgaatatgatattttattttgtgtatatccgagtttatggtttaagggaatgtagctcagttggtagagcatcgcatttgcattgcgagggtcgcgggttcgaaacctgtcatttccacaattatgaattgagagttataaaaaaggagagaaaatttctctccttttttagcgatttattaaataaataataaataaaagaaacaagaaacaaaaaattataggtcaaatgaacttaggcttacggtggagacctaggcacccagagacgaagaagggcgcagataccggcgaaacgctccggggagttggcaacaaactttgatccggagatccccgaatagggcaacctgtacaacttccaacagaattcataagttggaaagaggcaacccagtgaattgaaacatcttagtagctggaggaaaagaaagcaaacgcgattcccgtagtagcggcgagcgaaccgggaacagcctaaacccatttccattagggaatgggggtagtgggaagacattaatacgaaatagaaaataatttcaaactacgatattttctaaaaacgaaacagctgaatcctgtaccatagatggtgaaagtccagtagttgaaatgaaaaaaaaagaaaatgtcttattgtcttatcccgagtagcatgggacacgtggaatcccgtgtgaatcagcgaggaccacctcgtaaggctaaatactcctgggtgaccgatagtgaaatagtactgcgaaggaaaggtgaaaagtaccct

SEQ ID NO: 2 - DNA, 1980 bp, Artificial
  Prolidase expression construct for nuclear transformation of Dunaliella salina
actcacgcagcacaacccgctgcattttaaacaacgctgtccaacagggcctcctcactctcacactacctcaacaaacaaggggaagttggttactaatatcctggcacgcacagtcatccacactctctcaattaattcttttaacccccagtccacacacccttcacaaaacacacaatgcgcctggaaaagtttatccacctgctgggcgaacgtggttttgacggtgcactgatttccccaggcaccaacctgtattacctgacaggtctgcgtctgcacgaggtgggtgagcgcctggcaattctggcagtgtccgcagaaggcgactatcgctttctggctccttccctgtatgagaacgtggtgaacaacttccccgctacattctggcatgacggtgagaacccctacgctaagctgcgcgagattctggaggagctgggtatttccaaaggtcgtatcctgattgaggacaccatgcgcgcagattggctgatcggcattatgaagctgggtaagttcacttttcagccactgagcagcctgattaaggagctgcgcatgatcaaggacaaggaggaggtgaaaatgatggagcacgcatcccgcattgcagataaggtgtttgaggaaattctgacctgggatctgattggtatgaaggaacgcgaactggctctgaagatcgagctgctgatccgcgagctgagcgacggcatcgcattcgaacccattgtggcttccggcgaaaacgctgctaaccctcaccacgagcccggcgagcgtaagatccgcaagggtgatattatcatcctggattacggtgctcgctggaagggctattgttccgacattactcgcactattggcctgggcgaactggatgagcgcctggtgaaaatctatgaggtggtgaaggacgcacaggagtccgcatttaaagcagtgcgtgagggcattaaggcaaaggatgtggactcccgtgcacgcgaggtgatcagcaaggcaggttacggcgagtactttatccaccgtacaggccacggtctgggtctggacgtgcatgaagagccatatatcggtcccgatggtgaagtgatcctgaagaacggcatgaccttcactatcgagcctggcatctacgtgcccggcctgggcggtgtgcgtattgaagatgacattgtggtggatgagggcaagggccgtcgcctgacaaaggcagagcgtgaactgatcatcctgggcagcatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggaccaccaccaccaccaccactaagctgttagcacagcacttgccgcaatgcagtagctagggggggttgactctttcattggggtgtggaggggtgttaagagtaggagctgggctggtcatcacttttggtgacctcataactgttccataacaagcagacttgcatcctgtaagcatttgttgtggatgcctcctgcgtctagtctggcccatgatatatgcaatggagacagataaaggaaagtgagaagaagcttaggcttaaggctcatatatggctcaaggctcttgatcagacgagacagcacgatcagagtctgctattattctgtataagcagtacagaaaggatgg

SEQ ID NO: 3 - DNA, 4733 bp, Artificial
  Aminocyclase expression construct for nuclear transformation of Dunaliella salina
ttgacataaattgagtcctcaaggttaacaacctttaataaaagatgaatgcatagtctattttccattgaaaaatggttagtcataaatctctatgggattatgcggtttaatttgatgcaacgcaaagaaccttaccagggtttgacatgtctagaatttttcagaaatggaaaagtgcttcctttttatgaaaggaagaactagaacacaggtggtgcatggctgtcgtcagctcgtgcgttgacgtgtatggttaagtcctgcaacgagcgcaaccctcgtctttagttactttccaagttctctaaagagactgcgcgcgcaagcgctgaggaaggtgaggatgacgtcaagtcagcatgccccttacaccctgggcgacacgcgtaatacaatggttgggacaatcagaagctaccccgtaagggcacgccaatctgctaaactcaaccctagttcggattgtaggctgcaactcgcctacatgaagccggaatctatagtaatcgccagtcagctatatggcggtgaatacgttcctggtctctgtacacaccgcccgtcacatcaagaaaggtggtagtggattaagctactagtctaacctctctgaggaagacggtatccactccaagactactgatcatgatgaagtcgtaacaaggtaggggtactggaaggtgtccctggatcacctccttctttttaggaattttctatctgaccctcctaccaggagactaggacctttggtcctagcctcacggtaaacccaaagggtttaggcttaccttctcgtgaggggagctgacgcgttagatggaaaaataaaagtttttccatctataaagcgtaatgagatagaatcaataaaattatttaaaaggtgaccttaaccgaacgtaaattttacgttcggttaagtcacccttatatattcttatccaaaagaattttaatgggctattagctcagttggttagagcgcacccctgataagggtgaggtcagaagttcaaatcttctatagcccaccggacaaaacaactttagttaatatcgaagttttcaatatcctttaggatattgaaaatcttatagatttctataagaaaattctaatagagaagaaaatttacgaagtaaattttctattttcttcgataaattctgaaagagaagaaaatttactttgtaaatgttctattttcttccgaaaatttttttcgcggaaaaaacaaaagaaaaattttttttacgaaaaaaattttcccggacaaaactttgttttgtcctaaggaaaaaacaacaaagaaaattctgaaagaattttctttgttgttttgtccggcataaaaaaattttctttgtggttttgtcctaggaaaacccgtccccaaggcgggtccgttcgtccgtcgcgtgaggaggggggcacctttggttttttccgctgatggtttgtgtacgaggtcagcttccatgggcggggcggctgtttgcgatgctcttacgggcttaacacatgtatcttgtatagacctatttgtttatatcatttatcttaaaagaacaaaaaaaaaatttttggaggatcgtctactatggaaaaaaaaataacaggatatactacagtagacatttcacaatggcatcgtaaagaacatttcgaagcttttcaaagtgttgcacaatgtacttataaccaaacagttcaattagatattacagctttcttaaaaactgttaaaaaaaataaacacaaattttatccagcttttattcatattttagctcgtttaatgaatgctcatccagaatttcgtatggctatgaaagacggtgaattagttatttgggattcagttcatccttgttacactgtttttcacgaacaaacagaaactttctcatcattatggtcagaataccatgatgattttagacaatttttacacatttattcacaagatgtagcatgttatggtgaaaatttagcatattttcctaaaggttttattgaaaatatgttcttcgtttctgctaatccttgggtttctttcacttcttttgatttaaatgtagctaatatggataatttctttgcacctgtatttacaatgggtaaatattatacacaaggtgataaagttttaatgccattagcaattcaagtacatcatgctgtatgtgatggtttccacgtaggtcgtatgttaaacgaattacaacaatattgtgacgaatggcaaggtggtgctggaggatcgtctactatgttagttaaatcaatgaactttgatccattaaaagaagctgaaaaaattaaagatgaaattatttcatggagacgtgattttcatatgcaccctgaattaggttttgaagaagaacgtacttcaaaaattgtagaagaacatttacgtgaatggggttataaaattaaacgtgcaggtacaggaattattgcagatattggtgatggaggtaaaacaattgcattacgtgctgacatggatgctttaccaattcaagaagaaaatgatgttccatataaatctcgtgtaccaggaaaaatgcatgcttgtggtcacgatgcacatacagctatgttattaggtgcagctaaaattattgctgaacattcttctgaattagaaaataaagtaagattaattttccaaccagctgaagaaggaggtaacggtgcattaaaaatgattgaagctggtgctttagaaggtgtagatgctattttcggtattcacgtttgggctgaattaccttcaggtattgttggaattcgtgaaggtccatttttagctggagttggaaaatttattgcaaaaattattggtaaaggaggacatggtgcagctcctcacttttctattgatccaattcctgcagctgctgatgctgttttagcattacaacgtattgtagctcgtgaagtagacccattagaaagtgctgttgtaacagttggtaaaattcaaggtggtacagcttttaatgtaattccacaatatgttgaattagaaggtacatttcgtttctttacacaagaattaggtaaatttttagaacgtcgtattcgtgaaattattgaaaacactgctaaagcacataattgtaaagctgaagttaacacagaaattttaggtccaccaacaattaatgatgaaaaaatggtaaaatttgtagcagaaacagctaaagcattaggtttaaaagttggtgaagttagaaaaacattaggaggtgaagattttgcttattatttagaaaaagttcctggtgcttttattgcattaggtattcgtaatgaagaaaaaggtattatttatccacatcatcatccaaaatttgatgttgacgaagatgttttatatttaggaacagctttagaagttgctttagctttcaatttcaaaggacaccaccaccaccaccactaattctaaatacttaagaattaagtagacttgtctacttaattctttttaatagcatgggtggagtaaactccacccatgctattatttctaacaactataaaataagatttttttatnctaaatatcgaagttttccatatcgcagaagtatcacttctgcgagatctccgcgtttttctatagaaaaacgcggacatagaaaatcttcgagaaatgaatttttgtaatagaatctaatttaattacacaaatgtaaattacaaaaaggattgagcttcaggttcagctatataacctggttctgctatataaacaatataacttgaaatatcagtagatgaacaatttaatttatattaatagtcgatgtccttcaataacgaatcgtatttttatacggtatgttgatatttatatattctcttatatgttatgaatatatatcatatgttacgataacccccctctggggttattgtgagatctcgtttctaaaaaaacgaatatgatattttattttgtgtatatccgagtttatggtttaagggaatgtagctcagttggtagagcatcgcatttgcattgcgagggtcgcgggttcgaaacctgtcatttccacaattatgaattgagagttataaaaaaggagagaaaatttctctccttttttagcgatttattaaataaataataaataaaagaaacaagaaacaaaaaattataggtcaaatgaacttaggcttacggtggagacctaggcacccagagacgaagaagggcgcagataccggcgaaacgctccggggagttggcaacaaactttgatccggagatccccgaatagggcaacctgtacaacttccaacagaattcataagttggaaagaggcaacccagtgaattgaaacatcttagtagctggaggaaaagaaagcaaacgcgattcccgtagtagcggcgagcgaaccgggaacagcctaaacccatttccattagggaatgggggtagtgggaagacattaatacgaaatagaaaataatttcaaactacgatattttctaaaaacgaaacagctgaatcctgtaccatagatggtgaaagtccagtagttgaaatgaaaaaaaaagaaaatgtcttattgtcttatcccgagtagcatgggacacgtggaatcccgtgtgaatcagcgaggaccacctcgtaaggctaaatactcctgggtgaccgatagtgaaatagtactgcgaaggaaaggtgaaaagtaccct

SEQ ID NO: 4 - DNA, 2076 bp, Artificial
  Aminocyclase expression construct for nuclear transformation of Dunaliella salina
actcacgcagcacaacccgctgcattttaaacaacgctgtccaacagggcctcctcactctcacactacctcaacaaacaaggggaagttggttactaatatcctggcacgcacagtcatccacactctctcaattaattcttttaacccccagtccacacacccttcacaaaacacacaatgctggtgaaaagcatgaactttgatcccctgaaagaggcagagaagattaaagacgaaatcatctcctggcgccgcgacttccacatgcacccagagctgggtttcgaggaggagcgcacctccaagatcgtggaggaacatctgcgcgagtggggttataagattaaacgcgcaggtaccggcattattgctgatatcggcgacggtggtaagaccattgcactgcgcgcagacatggatgctctgcccattcaagaggagaacgatgtgccatacaagtcccgtgtgcctggtaaaatgcacgcttgcggccatgacgcacacactgcaatgctgctgggcgctgctaagattattgctgagcacagcagcgagctggaaaacaaggtgcgcctgatctttcagcctgcagaggaaggtggtaacggtgcactgaagatgattgaggctggtgcactggaaggtgtggatgcaatcttcggcatccacgtgtgggcagagctgcccagcggtatcgtgggtattcgtgaaggtcccttcctggcaggtgtgggtaagttcatcgctaagattatcggcaagggtggtcacggtgcagcaccccactttagcattgaccccatcccagcagcagcagacgctgtgctggcactgcagcgtattgtggcacgtgaagtggaccccctggagagcgctgtggtgaccgtgggcaagatccagggcggtaccgcttttaacgtgatcccccagtacgtggagctggagggtactttccgctttttcacccaggaactgggtaaattcctggaacgtcgtatccgtgagattatcgagaacaccgcaaaagctcacaactgcaaggcagaggtgaacaccgagattctgggcccccctacaatcaacgacgaaaaaatggtgaaatttgtggcagagaccgcaaaggcactgggcctgaaggtgggcgaagtgcgtaagacactgggcggcgaggattttgcttattacctggaaaaggtgccaggcgcttttattgcactgggtatccgcaacgaggagaagggtatcatttacccccaccaccaccccaagttcgacgtggacgaagacgtgctgtacctgggcaccgctctggaggtggcactggcatttaacttcaaaggtggcagcatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggaccaccaccaccaccaccactaagctgttagcacagcacttgccgcaatgcagtagctagggggggttgactctttcattggggtgtggaggggtgttaagagtaggagctgggctggtcatcacttttggtgacctcataactgttccataacaagcagacttgcatcctgtaagcatttgttgtggatgcctcctgcgtctagtctggcccatgatatatgcaatggagacagataaaggaaagtgagaagaagcttaggcttaaggctcatatatggctcaaggctcttgatcagacgagacagcacgatcagagtctgctattattctgtataagcagtacagaaaggatgg

SEQ ID NO: 5 - DNA, 3757 bp, Artificial
  Prolidase expression construct for nuclear transformation of Duneliella tertiolecta UTEX LB999
gcggccgcactcacgcagcacaacccgctgcattttaaacaacgctgtccaacagggcctcctcactctcacactacctcaacaaacaaggggaagttggttactaatatcctggcacgcacagtcatccacactctctcaattaattcttttaacccccagtccacacacccttcacaaaacacacaagatctatggccaagctgaccagcgccgtgcccgtcctgaccgctcgcgacgtggctggcgcggtggagttctggaccgaccgcctgggcttctcccgggacttcgtggaggacgatttcgcgggcgtggtgcgggacgacgtgaccctgttcattagcgcggtccaggaccaggtggtccccgataacaccctggcttgggtgtgggtgcgcggcctggacgagctgtacgccgagtggtccgaggtcgtcagcaccaacttccgcgacgcctccggccccgcgatgaccgagatcggcgagcagccctggggccgcgagttcgccctgcgcgaccccgccggcaactgcgtgcacttcgtggcggaggagcaggactaaccatgggctgttagcacagcacttgccgcaatgcagtagctagggggggttgactctttcattggggtgtggaggggtgttaagagtaggagctgggctggtcatcacttttggtgacctcataactgttccataacaagcagacttgcatcctgtaagcatttgttgtggatgcctcctgcgtctagtctggcccatgatatatgcaatggagacagataaaggaaagtgagaagaagcttaggcttaaggctcatatatggctcaaggctcttgatcagacgagacagcacgatcagagtctgctattattctgtataagcagtacagaaaggatgggctagcatgaagttcactagcttgcttttaggggggggtgcagaaggttttggagaaatggtggatgcactccttgctcacccttggctggactttcaggcgtctctgcacccaaacatcttcacttgaatgaactggatgaggaatttcctaaaggagcaacaacattgtcacccgaagagcatgggttcacacttatcactcaaaatcacgccttttaagtttgtgagatttctcgcattaccactttactttatacgtccattcctgcacacacccactcacacaccaccccagtcagacacaggtaccgtgagtgtgtgcgcaagcatttgcgcgctcgggtgagcaaggaaaatacagtgttgatgacagggacattagggcaggcaggcaagaaacgcacctcatgactctgcactgtatacatccttgctcagtcccaggtgcagcgcacacagcagcaagctctgcagccattgctcgtgcgctgcatgctcccaggaacatcttaatcgttgagggtgctgaacggaggagcagatgcacacatgggttggatgatgacacgcaagcaagcacacacgcaccgcatatgccgcctctcccttatctcggggggcccctcctcctctctcctgcccccatcttctctctcatcctcctccattttgcataggtgccagacgacaggccgctggttatccacgtcttctcacttcccctgcctccccaccttgcaccctcctctcctcgcaggtcgacatgcggctggagaagtttattcacctgctgggcgagcgcggcttcgacggcgccctgattagccccggcaccaacctctactacctcacgggcctgcgcctgcacgaggtcggcgagcgcctggcgatcctggccgtctccgccgagggcgactaccgcttcctggccccctccctgtacgagaacgtggtgaacaactttcccgccaccttttggcacgacggcgagaacccctacgccaagctgcgggagatcctggaggagctgggcatctccaagggccgcatcctcattgaggacaccatgcgggcggactggctgatcggcatcatgaagctgggcaagttcacgttccagcccctgtcctccctgattaaggagctgcggatgatcaaggacaaggaggaggtgaagatgatggagcacgcgagccgcatcgcggacaaggtgttcgaggagatcctgacctgggacctgatcggcatgaaggagcgcgagctggccctgaagatcgagctgctcattcgcgagctctccgatggcatcgcgttcgagcccattgtggcgtccggcgagaacgccgccaacccccaccacgagcccggcgagcgcaagatccgcaagggcgacatcatcatcctcgactacggcgcgcgctggaagggctactgctccgatattacccggaccatcggcctcggcgagctggacgagcgcctggtgaagatttacgaggtggtcaaggacgcccaggagtccgcgttcaaggccgtgagtatgtgtgctgcgtgattccagtgaattctgttggataagggagatgctcctggtcagcctccgtcttgtgccgtagtgctgtgtgcttctcctccttgcgtgatgccagtgttctcctctttactgcaatggtctcgcctttgcttgcccctatcctgtcctctgagttacaagagttgtcatgttagatgataaccattgtttgcagctctaatccctgtttctcccttcccttgcccctctccaggtgcgcgagggcatcaaggcgaaggacgtggacagccgcgcgcgcgaggtcattagcaaggcgggctacggcgagtacttcatccaccggaccggccacggcctcggcctggatgtgcacgaggagccctacatcggccccgacggcgaggtgatcctgaagaacggcatgaccttcacgatcgagcccggcatttacgtgcccggcctgggcggcgtgcgcatcgaggacgacatcgtggtggacgagggcaagggccgccgcctgaccaaggccgagcgcgagctcatcatcctgtaatctagagtgagtggaatgaataatgatgcagacagttttttgtcacatgtcagtggcctctccttagagcagtgtagctgtagtgagccatgtacatgtagccatgtactgcccagtaaaaattcatgagcaccctgggtgtcctcatctttttttaacgcctcttcctcggcatcctcaaccttagtcgaagttgactgattatatatatataccatttcttgtgttgaaatgatgttctgtgagtggtgaaaaaaagaatagtgtgtgtcagcaaaggtttttgtcatgcatgaactgaacatgcgttcacaacacatcccctgcagctcgaggcagctaccctgaagggtttagcctgacatagcctggacttgagtgcatcacctggcatgcgtcaatttatgtcatcgtttcccatacactcagctagtgtgtgttgaataaactccacaagcgaacacttgaactttggcgtgaactatgcctacaggcataatttttgtccacaatgcatgctcacagagggctgcagctgcatacatcctacatttcattgaaccatgtaggaagtgttgtgtgacacaaacactgcacacctcatttagttgttagccacctagctttttcgtttccccttgtttggatctttgcttgggaccacctggtctataccttggttctggtctaagggttccctttcattctgcaatgaatgctctgccgagacgtcggcaccattatagatgtaagcggccgc

(19) United States
(12) Patent Application Publication
(10) Pub. No.: US 2013/0323803 A1
(43) Pub. Date: Dec. 5, 2013

(54) METHODS AND COMPOSITIONS FOR THE PRODUCTION OF EXTREMOPHILE ENZYMES FROM GREEN MICROALGAE AND CYANOBACTERIA

(71) Applicants: Amy Michele Grunden, Holly Springs, NC (US); Heike Inge Ada Sederoff, Raleigh, NC (US)

(72) Inventors: Amy Michele Grunden, Holly Springs, NC (US); Heike Inge Ada Sederoff, Raleigh, NC (US)

(73) Assignee: North Carolina State University, Raleigh, NC (US)

(21) Appl. No.: 13/983,502

(22) PCT Filed: Feb. 3, 2012
(86) PCT No.: PCT/US12/23760

§ 371 Date: Aug. 19, 2013

Related U.S. Application Data
(60) Provisional application No. 61/439,490, filed on Feb. 4, 2011.

(51) Int. Cl. C12N 9/80; C12N 9/48

(52) U.S. Cl. CPC C12N 9/80; C12N 9/485
USPC 435/134; 435/189; 435/193; 435/195; 435/232; 435/233; 435/183; 536/23.2; 435/320.1; 435/476; 435/257.2

(57) ABSTRACT

The present invention relates to compositions and methods for stable transformation of green microalgae and for production of transgenic green microalgae and/or cyanobacteria that produce extremophile enzymes as co-products during the growth of the green microalgae and/or cyanobacteria for lipid biofuel production. Thus, the present invention provides nucleic acid constructs and methods of transformation useful in the production of stably transformed green microalgae and/or cyanobacteria expressing extremophile enzymes in combination with lipid production for biofuel.

### STATEMENT OF PRIORITY

This application claims the benefit, under 35 U.S.C. §119 (e), of U.S. Provisional Application No. 61/439,490, filed Feb. 4, 2011, the entire contents of which are incorporated by reference herein.

### STATEMENT OF GOVERNMENT SUPPORT

Aspects of the present invention were made with government support under National Science Foundation Grant No. 0937721 and Army Research Office Grant No. 44258-LS-SR Grunden. The United States Government has certain rights to this invention.

### FIELD OF THE INVENTION

The present invention relates to compositions and methods for stable transformation of microalgae and for production of transgenic green microalgae and cyanobacteria that produce extremophile enzymes as co-products during the growth of the green microalgae and cyanobacteria for lipid biofuel production.

### BACKGROUND

Enzyme based production of chemicals is a rapidly growing industrial sector that is primarily driven by the production and use of enzymes from microbial sources (Tang and Zhao, 2009; Otero and Nielsen, 2010). Thus, industrially important enzymes have been isolated from many types of microorganisms including from those organisms termed extremophiles, which are organisms that thrive in extreme environments. One of the advantages of extremophile enzymes is that they can function in extreme environments which are often present in the industrial setting. Extremophiles include Bacteria, Archaea and some animals. The types of extreme environments that these organisms have been isolated from include those that are extreme in temperature (thermophile, hyperthermophile, psychrophile), pH (acidophile, alkaliphile), salinity (halophile), metal ion concentrations, pressure and radiation levels (de Champdore et al., J. R. Soc. Interface 4:183-191 (2007)). The physical parameters that define some of these extreme environments include a temperature range of 55-80° C. for a thermophile, a temperature greater than 80° C. for a hyperthermophile, temperatures of −20° C. to 20° C. for a psychrophile, a salt concentration of greater than 2M salt for a halophile, a pH≦5 for an acidophile, a pH greater than 9 for an alkalophile, and a pressure of greater than 10 Mpa for a piezophile.

Examples of commercially valuable enzymes that have been isolated from extremophiles (i.e., extremozymes) have included, for example, Taq DNA polymerase from Thermus aquaticus, thermoactive α-amylases, β-amylases, lipases and proteases. Thus, many high value enzymes are produced by these organisms.

In recent years, industrial scale production of biofuels from macroalgal sources has received considerable attention. However, the relatively high cost of producing biofuels in these organisms has typically outweighed the economic value of the biofuel produced. (Scott et al. Curr. Opin. Biotechnol. 21:277-286 (2010); Williams and Laurens Energy Environ. Sci. 3:554-590 (2010).

Thus, the present invention addresses the previous shortcomings of the art by providing methods for the stable transformation of green microalgae wherein the transgenic green microalgae of the present invention can be used to produce extremophile enzymes and lipids useful in industrial biotechnology, thus providing high value industrial enzymes in addition to the production of lipid biofuels.

### SUMMARY OF THE INVENTION

The production of industrially important enzymes from extremophile organisms may be considered high-value co-products in green microalgae or cyanobacteria based biofuel production systems. Thus, the present invention provides methods and compositions for stable transformation of green microalgae and for production of transgenic green microalgae and cyanobacteria that produce extremophile enzymes as co-products during the growth of the green microalgae and cyanobacteria for lipid biofuel production.

In one aspect of the present invention, a nucleic acid construct is provided for plastid transformation of a green microalgae cell, the nucleic acid construct comprising in the following order from 5′ to 3′: (a) a left flanking sequence for homologous recombination (FS1); (b) a first promoter (P1); (c) a first enhancer sequence (EN1); (d) a heterologous nucleotide sequence encoding one or more extremophile enzymes (NSEE); (e) a first terminator (T1); (f) a right flanking sequence for homologous recombination (FS2); and wherein the one or more extremophile enzymes (NSEE) are modified for codon usage bias for the green microalgae cell; and F1 and F2 comprise nucleotide sequences that are homologous to the chloroplast genome of the green microalgae cell. In an additional aspect, the nucleic acid construct of the invention can further comprise a selection cassette comprising in the following order from 5′ to 3′: (a) a second promoter (P2); (b) a second enhancer sequence (EN2); (c) a nucleotide sequence for selection which confers resistance to a selection agent or encodes a selection protein (NSS); and (d) a second terminator (T2), wherein the NSS is modified for codon usage bias for the green microalgae cell and P2, EN2, NSS and T2 are operably located in the nucleic acid construct 3′ of FS1, and 5′ of P1 and wherein the selection cassette is operably located immediately downstream of FS1 and upstream of P1 or immediately downstream of T1 and upstream of FS2.

In further aspects of the invention, the selection cassette and the cassette comprising a heterologous nucleotide sequence encoding one or more extremophile enzymes (NSEE) are separate. Thus, in some exemplary embodiments, the present invention provides an expression cassette comprising the selection marker and a separate cassette comprising a heterologous nucleotide sequence encoding one or more extremophile enzymes, which can be used to co-transform the microalgae.

In other aspects of the invention, non-limiting examples of extremophile enzymes include an oxidoreductase, a transferase, a hydrolase, a lyase, an isomerase, a ligase, and the like, and any combination thereof.

In a further aspect, the present invention provides a method for stably transforming a green microalgae cell with a heterologous nucleotide sequence, the method comprising: propelling the heterologous nucleotide sequence at a green microalgae cell embedded in a gel at a velocity sufficient to pierce the cell wall (when present), and/or cell membrane and chloroplast membrane and deposit the heterologous nucleotide sequence within a chloroplast of the green microalgal cell; wherein the heterologous nucleotide sequence is incorporated into the green microalgae chloroplast genome, thereby producing a stably transformed green microalgae cell, wherein the heterologous nucleotide sequence is carried by a microprojectile and the heterologous nucleotide sequence is propelled at the microalgae cell by propelling the microprojectile at the green microalgae cell. In other aspects of the invention, the heterologous nucleotide sequence encodes one or more extremophile enzymes. Non-limiting examples of an extremophile enzyme include an oxidoreductase, a transferase, a hydrolase, a lyase, an isomerase, a ligase, or any combination thereof.

In additional aspects of the present invention, the green microalgae of the invention can have a cell wall. In other aspects of this invention, the green microalgae can be cell wall-less. Thus, non-limiting examples of the green microalgae of the present invention are green microalgae from the green microalgae families of Dunaliellaceae, Characiochloridaceae, Chlamydomonadaceae, Golenkiniaceae, Spondylomoraceae, Tetrabaenaceae,Volvocaceae, Haematococcaceae, Asteromonadaceae, Astrephomenaceae, Phacotaceae, Oocystaceae, Chlorellaceae, Eremosphaeraceae or Characiosiphonaceae, and the like.

The present invention further provides a transformed green microalgae cell produced by the methods described herein.

In addition, some aspects of the present invention provide a method for producing one or more extremophile enzymes, the method comprising: (a) culturing the transformed green microalgae cell of the present invention that expresses one or more extremophile enzymes; and (b) collecting the one or more extremophile enzymes from the green microalgae cell culture of (a), thereby producing one or more extremophile enzymes

In some additional aspects of the present invention, a method for producing lipids and extremophile enzymes in a green microalgae cell is provided, the method comprising: (a) culturing the transformed green microalgae cell of the present invention that expresses one or more extremophile enzymes, wherein the green microalgae cell further produces endogenous lipids; and (b) collecting the endogenous lipids and the one or more extremophile enzymes from the green microalgae cell culture of (a), thereby producing lipids and extremophile enzymes in a green microalgae cell.

In other aspects, the present invention provides a method for producing modified lipids and extremophile enzymes in a green microalgae cell, the method comprising: (a) culturing the stably transformed green microalgae cell of the present invention expressing one or more enzymes for modifying lipids and one or more (other) extremophile enzymes, for a time sufficient for the one or more enzymes for modifying lipids to modify the lipids produced in the green microalgae cell; and (b) collecting the modified lipids and the one or more extremophile enzymes in the green microalgae cell culture of (a), thereby producing modified lipids and extremophile enzymes in a green microalgae cell.

The foregoing and other objects and aspects of the present invention are explained in detail in the drawings and specification set forth below.

### BRIEF DESCRIPTION OF THE DRAWINGS

FIG. 1 shows exemplary flanking sequences for integration between trnI/trnA.

### DETAILED DESCRIPTION

This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention. All publications, patent applications, patents, and other references mentioned herein are incorporated by reference in their entirety. References to techniques employed herein are intended to refer to the techniques as commonly understood in the art, including variations on those techniques or substitutions of equivalent techniques that would be apparent to one of skill in the art.

All patents, patent publications, non-patent publications and sequences referenced herein are incorporated by reference in their entireties.

Green microalgae and cyanobacteria are ideal host systems for recombinant expression of value-enhanced co-products from biofuel systems because they can grow rapidly and to high densities, can produce high levels of lipids for biofuel conversion and are not in competition with crops for arable land. These organisms can also be used to produce other high value products such as industrial enzymes.

For enzymes to be useful in many industrial processes, the enzymes must have good catalytic activity under the desired processing conditions, which depending on the industrial process can be very harsh, and these enzymes must have the ability to maintain activity in these processing conditions for extended periods of time (Vieille and Zeikus. Microbio. Mol. Biol. Rev. 65:1-43 (2001)). Extremophile enzymes provide significant biotechnological advantages compared to their mesophilic counterparts. For instance, they usually have higher resistance to chemical denaturants and extremes in pH (Haki et al. Bioresour Technol 89:17-34 (2003)). Extremophile enzymes are also typically able to withstand high substrate concentrations without losing catalytic efficiency, and the enzyme reaction rates are faster and less susceptible to microbial contamination under industrial processing conditions (Li et al. Biotechnol Adv 23:271-281 (2005)). Higher reaction rates catalyzed by these enzymes lead to accelerated reactions with shorter conversion periods and a higher substrate to product conversion, which saves industrial processing time (van den Burg. Current Opinion in Microbiology 6:213-218 (2003)). Another benefit to extremozymes that are made recombinantly in mesophilic expression hosts, is that these proteins generally have limited activity under the mesophilic host growth conditions, and therefore, the enzymes do not typically interfere with host metabolism (Grunden et al. Expression of Extremophilic Proteins In F Baneyx, ed, Expression Technologies: Current Status and Future Trends. Horizon Scientific Press, Norfolk, pp 1-84 (2004)).

Extremophile enzymes are produced by extremophile organisms, which are organisms that thrive in extreme environments. Extremophiles include, for example, Bacteria, Archaea and some animals. The types of extreme environments that these organisms have been isolated from include those that are extreme in temperature (thermophile, hyperthermophile, psychrophile), pH (acidophile, alkaliphile), salinity (halophile), metal ion concentrations, pressure and radiation levels (de Champdore et al., J. R. Soc. Interface 4:183-191 (2007)). The physical parameters that define some of these extreme environments include a temperature range of 55-80° C. for a thermophile, a temperature greater than 80° C. for a hyperthermophile, temperatures of −20° C. to 20° C. for a psychrophile, a salt concentration of greater than 2M salt for a halophile, a pH≦5 for an acidophile, a pH greater than 9 for an alkalophile, and a pressure of greater than 10 Mpa for a piezophile.

Thus, provided herein is an expression platform for the production of biotechnologically important extremozymes as co-products in biofuel-producing green microalgae and cyanobacteria.

In representative embodiments, the extremozymes and their respective coding sequences originate from Archaea. Archaea belong to a different kingdom than green microalgae or cyanobacteria. As far as the inventors are aware, Archaea genes have never been transformed into green microalgae or cyanobacteria. An analysis of codon usage bias shows that there are distinct differences between codon usage in the nucleic acids of green microalgae chloroplasts and cyanobacteria. In particular, it is noted that the frequency of use of particular codons in genes from extremophile Archaea are dramatically different when compared to the use of these same codons in green microalgae chloroplast genes and cyanobacterial genes. Thus, because of the difference in codon usage, transformation of green microalgae chloroplasts or cyanobacteria with Archaea coding sequences would not be expected to produce any protein.

In some embodiments, enzymes from other extremophile organisms, in addition to Archaea, can be used including those from fungi, bacteria, and plants.

Accordingly, a first aspect of the present invention provides a nucleic acid construct for plastid transformation of a green microalgae cell, the nucleic acid construct comprising in the following order from 5′ to 3′: (a) a left flanking sequence for homologous recombination (FS1); (b) a first promoter (P1); (c) a first enhancer sequence (EN1); (d) a heterologous nucleotide sequence encoding one or more extremophile enzymes (NSEE); (e) a first terminator (T1); and (f) a right flanking sequence for homologous recombination (FS2); wherein the one or more extremophile enzymes (NSEE) are modified for codon usage bias for the green microalgae cell; and the left and right flanking sequences for homologous recombination comprise nucleotide sequences that are homologous to the chloroplast genome of the green microalgae cell. In an additional aspect, the nucleic acid construct of the invention can further comprise a selection cassette comprising in the following order from 5′ to 3′: (a) a second promoter (P2); (b) a second enhancer sequence (EN2); (c) a nucleotide sequence for selection which confers resistance to a selection agent or encodes a selection protein (NSS); and (d) a second terminator (T2), wherein the NSS is modified for codon usage bias for the green microalgae cell and P2, EN2, NSS and T2 are operably located in the nucleic acid construct 3′ of FS1, and 5′ of P1 and wherein the selection cassette is operably located immediately downstream of FS1 and upstream of P1 or immediately downstream of T1 and upstream of FS2.

In additional embodiments, the nucleotide sequence for selection, which confers resistance to a selection agent or encodes a selection protein (NSS), and the nucleotide sequence encoding one or more extremophile enzymes (NSEE) can be on separate expression/transformation cassettes or nucleic acid constructs. In some embodiments, one or more extremophile enzymes can also be on separate expression cassettes. The separate expression cassettes are inserted into separate plasmid vectors. Any plasmid vector can be used that can be propagated in bacteria (e.g., E. coli). The plasmids can then be propagated separately in bacteria and mixed together before transformation of the microalgae, thereby co-transforming the two plasmids into the microalgae cells. Methods for co-transformation are known in the art (Coragliotti et al. (2011) Molecular Biotechnology 48: 60-75 and Poulsen et al. (2006) Journal of Phycology 42: 1059-1065).

In some embodiments, the nucleic acid constructs for transformation of the microalgae do not comprise the plasmid backbone, and thus are provided as minimal nucleic acid constructs/linear expression constructs. The plasmid backbone can be removed using restriction endonuclease digestion followed by gel purification of the nucleic acid construct as is well-known in the art.

Accordingly, in some embodiments, a nucleic acid construct is provided for chloroplast co-transformation of a microalgae cell, comprising in the following order from 5′ to 3′ as follows: left flanking sequence-promoter-enhancer sequence-nucleotide sequence of interest (e.g., nucleotide sequence encoding a selection marker or a nucleotide sequence encoding one or more extremophile enzymes and/or one or more lipid modifying enzymes)-terminator-right flanking sequence. The flanking sequences for the cassettes comprising a nucleotide sequence encoding a selection marker and those for the cassettes comprising the nucleotide sequence of interest are constructed to target different insertion sites in the chloroplast. The promoters and terminators used in the construction of the separate expression cassettes to be used in co-transformation can be identical or different.

In some embodiments, the heterologous nucleotide sequence encoding one or more extremophile enzymes can be introduced into the green microalgae cell using, for example, a minichromosome vector.

Any promoter can be used in the nucleic acid constructs of the present invention that can initiate transcription in a cell or in a chloroplast of a green microalgae or a cyanobacteria, and thus, drive expression of an operably associated nucleotide sequence. Thus, for example, any promoter from any chloroplast gene can be used in the nucleic constructs of this invention. In some embodiments, the promoter can be from a highly expressed chloroplast gene.

As used herein, the term “promoter” refers to a region of a nucleotide sequence that incorporates the necessary signals for.the efficient expression of a coding sequence operably associated with the promoter. This may include sequences to which an RNA polymerase binds, but is not limited to such sequences and can include regions to which other regulatory proteins bind, together with regions involved in the control of protein translation and can also include coding sequences. Furthermore, a “promoter” of this invention is a promoter (e.g., a nucleotide sequence) capable of initiating transcription of a nucleic acid molecule in a cell of a green microalgae or cyanobacteria.

In some embodiments of the invention, the promoters in the nucleic acid constructs can be species specific for the particular green microalgae being transformed. In some embodiments of the present invention, the promoters, P1 and P2 can be the same promoter. In other embodiments of the present invention, P1 and P2 can be different promoters. Non-limiting examples of the first and/or second promoter, P1 and P2, include the promoter of the σ⁷⁰-type plastid rRNA gene (Prrn), the promoter of the psbA gene (encoding the photosystem-II reaction center protein D1) (PpsbA), the promoter of the psbD gene (encoding the photosystem-II reaction center protein D2) (PpsbD), the promoter of the psaA gene (encoding an apoprotein of photosystem I) (PpsaA), the promoter of the ATPase alpha subunit gene (PatpA), and promoter of the RuBisCo large subunit gene (PrbcL), and any combination thereof (See, e.g., De Cosa et al. Nat. Biotechnol. 19:71-74 (2001); Daniell et al. BMC Biotechnol. 9:33 (2009); Muto et al. BMC Biotechnol. 9:26 (2009); Surzycki et al. Biologicals 37:133-138 (2009)). In some embodiments, the first and second promoters of the nucleic acid construct are the promoter of the σ⁷⁰-type plastid rRNA gene (Prrn).

Any terminator from any highly expressed chloroplast gene can be used in the nucleic constructs of this invention. A terminator of the present invention can be a terminator in the respective gene from which a promoter is chosen. Thus, similar to the promoters of the present invention, terminators useful with the present invention can be species specific for the green microalgae to be transformed. Non-limiting examples of terminators of the nucleic acid constructs of the present invention (T1 and T2) include the terminator of the psbA gene (TpsbA), the terminator of the psaA gene (encoding an apoprotein of photosystem I) (TpsaA), the terminator of the psbD gene (TpsbD), RuBisCo large subunit terminator (TrbcL), the terminator of the σ⁷⁰-type plastid rRNA gene (Trrn), and the terminator of the ATPase alpha subunit gene (TatpA). In some embodiments of the present invention, the terminators, T1 and T2 can be the same terminator. In other embodiments of the present invention, T1 and T2 can be different terminators. In additional embodiments of this invention, the terminators can be from different genes.

As noted above, promoters and terminators from any source can be used with the present invention. For example, in addition to the promoters and terminators described above, promoters and terminators can be derived from other microalgae (Hallman and Wodniok. Plant Cell Reports 25: 582-591 (2006)), or from plants or viruses (Geng et al., J. Appl. Phycol. 15: 451-456 (2003)). In some embodiments, specific elements from promoters and terminators from various sources can be combined into a single promoter or terminator, e.g., for higher expression level of the gene being transformed into the microalgae (Id.).

The left and right flanking sequences of the nucleic acid constructs of the present invention facilitate homologous recombination of the nucleic acid construct (e.g., expression cassette) into the chloroplast genome by REC1 (Nakazato et al. Biosci. Biotechnol. Biochem. 67: 2608-2613 (2003)) and as such these sequences are specific to the species of green microalgae to be transformed. Thus, species specific loci and sequences are identified for use as flanking sequences for homologous recombination of expression cassettes into the plastid genome of the green microalgae. The flanking sequences can be derived from any sequence in the respective green microalgae chloroplast genome. Once the flanking sequences are chosen, they can be amplified using sequence specific or conserved primers. Thus, in some embodiments of the present invention, the left flanking sequence can comprise about 250 base pairs to about 5000 base pairs that are selected from the nucleotides that are 5′ to and adjacent to the chosen integration site. In further embodiments of the present invention, the right flanking sequence can comprise about 250 base pairs to about 5000 base pairs that are selected from the nucleotides that are 3′ to and adjacent to the chosen integration site. Thus, the right and/or left flanking sequences can be about 250 base pairs, about 275 base pairs, about 300 base pairs, about 325 base pairs, 350 base pairs, about 375 base pairs, about 400 base pairs, about 425 base pairs, about.450 base pairs, about 475 base pairs, about 500 base pairs, about 525 base pairs, about 550 base pairs, about 575 base pairs, about 600 base pairs, about 625 base pairs, about 650 base pairs, about 675 base pairs, 700 base pairs, about 725 base pairs, about 750 base pairs, about 800 base pairs, about 825 base pairs, about 850 base pairs, about 875 base pairs, 900 base pairs, about 925 base pairs, about 950 base pairs, about 975 base pairs; about 1000 base pairs, about 1050 base pairs, about 1100 base pairs, about 1150 base pairs, about 1200 base pairs, about 1250 base pairs, about 1300 base pairs, about 1350 base pairs, about 1400 base pairs, about 1450 base pairs, about 1500 base pairs, about 1550 base pairs, about 1600 base pairs, about 1650 base pairs, about 1700 base pairs, about 1750 base pairs, about 1800 base pairs, about 1850 base pairs, about 1900 base pairs, about 1950 base pairs, about 2000 base pairs, about 2050 base pairs, about 2100 base pairs, about 2150 base pairs, about 2200 base pairs, about 2250 base pairs, about 2300 base pairs, about 2350 base pairs, about 2400 base pairs, about 2450 base pairs, about 2500 base pairs, about 2550 base pairs, about 2600 base pairs, about 2650 base pairs, about 2700 base pairs, about 2750 base pairs, about 2800 base pairs, about 2850 base pairs, about 2900 base pairs, about 2950 base pairs, about 3000 base pairs, about 3050 base pairs, about 3100 base pairs, about 3150 base pairs, about 3200 base pairs, about 3250 base pairs, about 3300 base pairs, about 3350 base pairs, about 3400 base pairs, about 3450 base pairs, about 3500 base pairs, about 3550 base pairs, about 3600 base pairs, about 3650 base pairs, about 3700 base pairs, about 3750 base pairs, about 3800 base pairs, about 3850 base pairs, about 3900 base pairs, about 3950 base pairs, about 4000 base pairs, about 4050 base pairs, about 4100 base pairs, about 4150 base pairs, about 4200 base pairs, about 4250 base pairs, about 4300 base pairs, about 4350 base pairs, about 4400 base pairs, about 4450 base pairs, about 4500 base pairs, about 4550 base pairs, about 4600 base pairs, about 4650 base pairs, about 4700 base pairs, about 4750 base pairs, about 4800 base pairs, about 4850 base pairs, about 4900 base pairs, about 4950 base pairs, about 5000 base pairs, and the like, or any range therein. In further embodiments, the left flanking sequence comprises at least about 1000 base pairs that are selected from the nucleotides that are 5′ to and adjacent to the chosen integration site. In other embodiments, the right flanking sequence comprises at least about 1000 base pairs that are selected from the nucleotides that are 3′ to and adjacent to the chosen integration site.

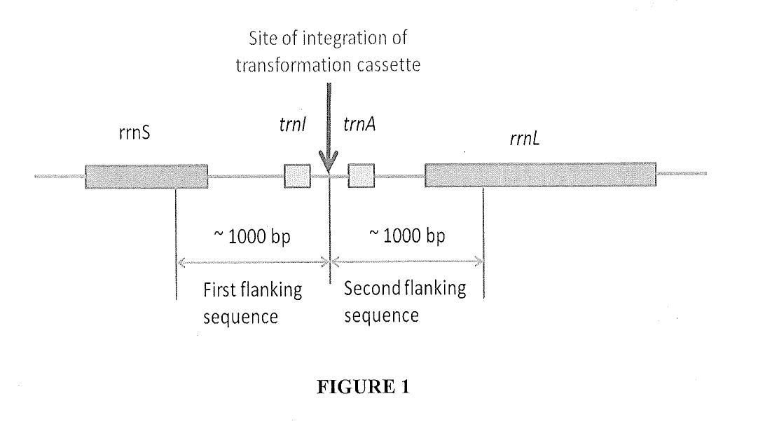

In some embodiments, the transcriptionally active intergenic region between trnI and trnA is selected as the integration site for the nucleic acid construct of the present invention. Therefore, in some embodiments of the present invention, the left flanking sequence is about 1000 base pairs in length and is derived from the nucleotide sequence that is 5′ and adjacent to this chosen integration site and encompasses the region encoding trnI, the intergenic region between trnI and rrnS and optionally a, portion of the nucleotide sequence encoding the 3′ end of rrnS and the right flanking sequence is about 1000 base pairs in length and is derived from the nucleotide sequence that is 3′ and adjacent to this chosen integration site and encompasses the region encoding trnA, the intergenic region between trnA and rrnL, and optionally a portion of the nucleotide sequence encoding the 5′ end of rrnL (See, for example, FIG. 1).

In some embodiments of the invention, the nucleic acid construct comprises an enhancer sequence. Enhancer sequences can be derived from any intron from any expressed green microalgae chloroplast gene for the nucleic acid constructs for chloroplast transformation of green microalgae. In some embodiments, the enhancer sequences can be derived from any intron from any highly green microalgae chloroplast gene. In other embodiments, enhancer sequences can be derived from any intron from any green microalgae nuclear gene for use with the nucleic acid constructs for nuclear transformation. In still further embodiments of this invention, an enhancer sequence usable with the present invention can include, but is not limited to, a nucleotide sequence encoding a ribosome binding site (e.g., ggagg). Accordingly, in some embodiments of the present invention, the nucleic acid constructs comprise first and/or second enhancer sequences, wherein the first and/or second enhancer sequences are the nucleotide sequence of, for example, ggagg.

As described herein, the nucleotide sequences encoding the extremophile enzymes and the nucleotide sequences for selection are modified for codon usage bias using species specific codon usage tables. When these nucleotide sequences are to be expressed in chloroplasts, the codon usage tables are generated based on a sequence analysis of the expressed chloroplast genes for the green microalgae species of interest. In some embodiments, the codon usage tables for nucleotide sequences to be expressed in chloroplasts are generated based on a sequence analysis of the most highly expressed chloroplast genes for the green microalgae species of interest. When the nucleotide sequences are to be expressed in the nucleus, the codon usage tables are generated based on a sequence analysis of expressed nuclear genes for the green microalgae species of interest. In some embodiments, the codon usage tables for nucleotide sequences that are to be expressed in the nucleus are generated based on a sequence analysis of highly expressed nuclear genes for the green microalgae species of interest. The modifications for the nucleotide sequences for selection are determined by comparing the species specific codon usage table with the codons present in the native nucleotide sequences. In those embodiments in which each of the codons in the native nucleotide sequence for selection are sufficiently used, then no modifications are needed (e.g., a frequency of more than 30% for a codon used for a specific amino acid in that species would indicate no need for modification). In other embodiments, wherein up to 3 nucleotides have to be modified in the nucleotide sequence, site-directed mutagenesis can be used according to methods known in the art (Zheng et al. Nucleic Acids Res. 32:e115 (2004); Dammai, Meth. Mol. Biol 634:111-126 (2010); Davis and Vierstra. Plant Mol. Biol. 36(4): 521-528 (1998)). In still other embodiments, wherein more than three nucleotide changes are necessary, a synthetic nucleotide sequence can be generated using the same codon usage as the highly expressed chloroplast genes (or the highly expressed nuclear genes) that were used to develop the codon usage table.

In some embodiments, a nucleic acid construct of the present invention can further comprise an origin of replication (ori) derived, e.g., from the chloroplast of the microalgae cell. In some embodiments, the origin of replication can be specific for the species of microalgae to be transformed with the nucleic acid construct.

In a further aspect of the invention, a nucleic acid construct is provided for nuclear transformation of a green microalgae cell, comprising in the following order from 5′ to 3′: (a) a promoter; (b) a heterologous nucleotide sequence encoding one or more extremophile enzymes (NSEE); and (c) a terminator, wherein the one or more extremophile enzymes (NSEE) are modified for codon usage bias for the green microalgae cell. In an additional aspect, the nucleic acid construct for nuclear transformation can further comprise a nucleotide sequence for selection which confers resistance to a selection agent or encodes a selection protein (NSS), wherein the NSS is modified for codon usage bias for the green microalgae cell and is operably located 3′ to the promoter and 5′ to the NSEE. In some embodiments, the nucleotide sequence for selection can be present on a different nucleic acid construct from the nucleotide sequences encoding the one or more extremophile enzymes.

As described above, any promoter that can initiate transcription in a cell of a green microalgae or a cyanobacteria can be used in the nucleic acid constructs of the present invention. In some embodiments of the invention, the promoters of the nucleic acid constructs can be species specific for the green microalgae being transformed. Non-limiting examples of a promoter useful in the nucleic acid construct for nuclear transformation include the promoter of the RubisCo small subunit gene 1 (PrbcS1), the promoter of the actin gene (Pactin), the promoter of the nitrate reductase gene (Pm) and the promoter of duplicated carbonic anhydrase gene 1 (Pdac1) (See, Walker et al. Plant Cell Rep. 23:727-735 (2005); Li et al. Gene 403:132-142 (2007); Li et al. Mol Biol. Rep. 37:1143-1154 (2010)). PrbcS1 and Pactin are constitutive promoters and Pnr and Pdca1 are inducible promoters. Pnr is induced by nitrate and repressed by ammonium (Li et al. Gene 403:132-142 (2007)) and Pdca1 is induced by salt (Li et al. Mol Biol. Rep. 37:1143-1154 (2010)). These promoters and other promoters can be identified in and isolated from green microalgae to be transformed or from other organisms and then inserted into the nucleic acid construct to be used in transformation of the green microalgae cell.

Similar to the promoters useful with a nucleic acid construct for nuclear transformation of a green microalgae cell, a terminator useful with the present invention can be any terminator functional in a cell of a green microalgae or a cyanobacteria. Further, terminators useful with nucleic acid constructs of the present invention can be specific for the species of green microalgae to be transformed. In addition, in some embodiments, the terminators can be derived from the same gene from which the promoter is selected. Non-limiting examples of terminators of the nucleic acid constructs of the present invention include the terminator of the RubisCo small subunit gene 1 (TrbcS1), the terminator of the actin gene (Tactin), the terminator of the nitrate reductase gene (Tnr), and the terminator of duplicated carbonic anhydrase gene 1 (Tdac1). These and other terminators can be identified in and isolated from the green microalgae to be transformed (and other organisms) and then inserted into the nucleic acid construct to be used in transformation of the green microalgae cell.

As noted above, promoters and terminators from any source can be used with the present invention. Thus, for example, in addition to the promoters and terminators described above, promoters and terminators can be derived from other microalgae, or from plants or viruses. In some embodiments, specific elements from promoters and terminators from various sources can be combined into a single promoter or terminator for higher expression level of the gene being transformed into the microalgae.

In some embodiments, the nucleic acid constructs of the present invention for chloroplast and/or nuclear transformation comprise a nucleotide sequence for selection which confers resistance to a selection agent or encodes a selection protein (NSS), which can be used to select a transformed green microalgae cell and/or a transformed cyanobacteria cell. As used herein, “selectable marker” means a nucleotide sequence that when expressed imparts a distinct phenotype to the transformed green microalgae cell or transformed cyanobacteria cell expressing the marker and thus allows such transformed green microalgae cell or transformed cyanobacteria cell to be distinguished from those that do not have the marker. Such a nucleotide sequence may encode either a selectable or screenable marker, depending on whether the marker confers a trait that can be selected for by chemical means, such as by using a selective agent (e.g, an antibiotic, herbicide, or the like), or on whether the marker is simply a trait that one can identify through observation or testing, such as by screening (e.g., GUS, green fluorescent protein). Of course, many examples of suitable selectable markers are known in the art and can be used in the expression cassettes described herein.

Some non-limiting examples of nucleotide sequences that can be used for selection (NSS) include a nucleotide sequence encoding aadA (i.e., spectinomycin and streptomycin resistance), a nucleotide sequence encoding neo (i.e., kanamycin resistance), a nucleotide sequence encoding aphA6 (i.e., kanamycin resistance), a nucleotide sequence encoding nptlI (i.e., kanamycin resistance), a nucleotide sequence encoding bar (i.e., phosphinothricin resistance), a nucleotide sequence encoding cat (i.e., chloramphenicol resistance), a nucleotide sequence encoding Sh ble (bleomycin resistance (Streptoalloteichus hindustanus)), a nucleotide sequence encoding badh (i.e., betaine aldehyde resistance), a nucleotide sequence encoding egfp, (i.e., enhanced green fluorescence protein), a nucleotide sequence encoding gfp (i.e., green fluorescent protein), a nucleotide sequence encoding luc (i.e., luciferase), a nucleotide sequence encoding ble (i.e. bleomycin resistance), a nucleotide sequence encoding ereB (i.e. erythromcyin resistance) (WO/2011/034863), a nucleotide sequence encoding aphVIII (i.e., paromomycin resistance) (Hallman and Wodniok. Plant Cell Reports 25: 582-591 (2006)), a nucleotide sequence encoding nat (i.e., nourseothricin resistance) (Poulsen et al. J Phycol, 42: 1059-1065 (2006)), and any combination thereof. In some embodiments of the invention, these nucleotide sequences can be modified for codon usage bias for the green microalgae cell that is to be transformed.

Further examples of selectable markers useful with the present invention include, but are not limited to, a nucleotide sequence encoding an altered 5-enolpyruvylshikimate-3-phosphate (EPSP) synthase, which confers resistance to glyphosate (Hinchee et al. (1988) Biotech. 6:915-922); a nucleotide sequence encoding a nitrilase such as bxn from Klebsiella ozaenae that confers resistance to bromoxynil (Stalker et al. (1988) Science 242:419-423); a nucleotide sequence encoding an altered acetolactate synthase (ALS) that confers resistance to imidazolinone, sulfonylurea or other ALS-inhibiting chemicals (EP Patent Application No. 154204); a nucleotide sequence encoding a methotrexate-resistant dihydrofolate reductase (DHFR) (Thillet et al. (1988) J. Biol. Chem. 263:12500-12508); a nucleotide sequence encoding a dalapon dehalogenase that confers resistance to dalapon; a nucleotide sequence encoding a mannose-6-phosphate isomerase (also referred to as phosphomannose isomerase (PMI)) that confers an ability to metabolize mannose (U.S. Pat. Nos. 5,767,378 and 5,994,629); a nucleotide sequence encoding an altered anthranilate synthase that confers resistance to 5-methyl tryptophan; and/or a nucleotide sequence encoding hph that confers resistance to hygromycin.

Additional selectable markers include, but are not limited to, a nucleotide sequence encoding β-glucuronidase or uidA (GUS) that encodes an enzyme for which various chromogenic substrates are known; an R-locus nucleotide sequence that encodes a product that regulates the production of anthocyanin pigments (red color) in plant tissues (Dellaporta et al., “Molecular cloning of the maize R-nj allele by transposon-tagging with Ac” 263-282 In: Chromosome Structure and Function: Impact of New Concepts, 18th Stadler Genetics Symposium (Gustafson & Appels eds., Plenum Press 1988)); a nucleotide sequence encoding β-lactamase, an enzyme for which various chromogenic substrates are known (e,g., PADAC, a chromogenic cephalosporin) (Sutcliffe (1978) Proc. Natl. Acad. Sci. USA. 75:3737-3741); a nucleotide sequence encoding xylE that encodes a catechol dioxygenase (Zukowsky et al. (1983) Proc. Natl. Acad. Sci. USA 80:1101-1105); a nucleotide sequence encoding tyrosinase, an enzyme capable of oxidizing tyrosine to DOPA and dopaquinone, which in turn condenses to form melanin (Katz et al. (1983) J. Gen. Microbiol. 129:2703-2714); a nucleotide sequence encoding β-galactosidase, an enzyme for which there are chromogenic substrates; a nucleotide sequence encoding luciferase (lux) that allows for bioluminescence detection (Ow et al. (1986) Science 234:856-859); or a nucleotide sequence encoding aequorin which may be employed in calcium-sensitive bioluminescence detection (Prasher et al. (1985) Biochem. Biophys. Res. Comm. 126:1259-1268); or any combination thereof. One of skill in the art is capable of choosing a suitable selectable marker for use in an expression cassette of this invention.

In some embodiments of the present invention, the nucleic acid constructs for nuclear transformation of a microalgal cell comprise no introns. In other embodiments, the nucleic acid constructs for nuclear transformation of a microalgal cell can comprise at least one intron. In still other embodiments, the at least one intron and the promoter and the terminator can be derived from the same gene or from different genes. Thus, in some embodiments of this invention, each of the at least one intron and the promoter and the terminator can be derived from the same gene.

In further embodiments, the nucleic acid constructs for nuclear transformation can comprise one to ten introns (e.g., 1, 2, 3, 4, 5, 6, 7, 8, 9, or 10). In some embodiments of this invention, the one to ten introns can be derived from the same gene or they can be derived from different genes.

In other embodiments, the nucleic acid constructs for nuclear transformation can comprise one to ten introns (e.g., 1, 2, 3, 4, 5, 6, 7, 8, 9, or 10), wherein the one to ten introns and the promoter and the terminator of the nucleic acid constructs of the invention are each derived from the same gene. As would be understood by those of skill in the art, the introns as used herein comprise the sequences required for self excision and are incorporated into the nucleic acid constructs in frame.

An intron of this invention can be used either as a spacer to separate multiple protein-coding sequences in one nucleic acid construct, or an intron can be used inside one protein-coding sequence to stabilize the mRNA. If they are used within a protein-coding sequence, they are inserted “in-frame” with the excision sides included.

Non-limiting examples of introns useful in the present invention can be introns from the RuBisCO small subunit (rbcS) gene, the RuBisCO large subunit (rbcL) gene, the actin gene, the nitrate reductase gene (nr), the duplicated carbonic anhydrase gene 1 (Tdac1), the psbA gene, the atpA gene, and any combination thereof.

In other embodiments of the present invention, the nucleic acid construct for nuclear transformation of green microalgae can comprise three introns, intron 1 (IN1), intron 2 (IN2), and intron 3 (IN3). In some embodiments, the three introns and the promoter and the terminator of the nucleic acid constructs are derived from the same gene. Accordingly, in some embodiments, the present invention provides a nucleic acid construct for nuclear transformation of a green microalgae cell, the nucleic acid construct comprising in the following order from 5′ to 3′: (a) a promoter; (b) a first intron (IN1); (c) a heterologous nucleotide sequence encoding one or more extremophile enzymes (NSEE); (d) a second intron (IN2); (e) a heterologous nucleotide sequence for selection which confers resistance to a selection agent or encodes a selection protein (NSS); (f) a third intron (IN3) and (g) a terminator, wherein the one or more extremophile enzymes (NSEE) and the NSS are modified for codon usage bias for the green microalgae cell. In a still further embodiment, the heterologous nucleotide sequence (NSEE) can have an intron inserted within it (See, e.g., SEQ ID NO:5)

In some embodiments of the present invention, the microalgae can be any green microalgae (i.e., Chlorophyceae). In other embodiments, the microalgae of the present invention can be a marine green microalgae. In still other embodiments, the green microalgae of this invention can be a freshwater green microalgae. In additional embodiments of this invention, the green microalgae cell can be a green microalgae cell that has a cell wall. In other embodiments, the green microalgae cell is cell wall-less. This invention is further envisioned to encompass green microalgae that are cell wall-deficient mutants (see, e.g., Chlamydomonas rheinhardtii cell wall-deficient mutant CW-15; Davies and Plaskitt, Genet. Res. 17: 33-43. (1971)).

Thus, in further embodiments, the green microalgae cell can be from the family Dunaliellaceae, the family Characiochloridaceae, the family Chlamydomonadaceae, the family Golenkiniaceae, the family Spondylomoraceae, the family Tetrabaenaceae, the family Volvocaceae, the family Haematococcaceae, the family Asteromonadaceae, the family Astrephomenaceae, the family Phacotaceae, the family Oocystaceae, the family Chlorellaceae, the family Eremosphaeraceae or the family Characiosiphonaceae, or any combination thereof.

In other embodiments, the green microalgae cell can be of a green microalgae having no cell walls from the family Dunaliellaceae, the family Asteromonadaceae, or the family Characiosiphonaceae.

In still other embodiments, the green microalgae cell can be of a green microalgae having cell walls from the family Characiochloridaceae, the family Chlamydomonadaceae, the family Golenkiniaceae, the family Spondylomoraceae, the family Tetrabaenaceae, the family Volvocaceae, the family Haematococcaceae, the family Astrephomenaceae, the family Phacotaceae, the family Oocystaceae, the family Chlorellaceae, the family Eremosphaeraceae or the family Characiosiphonaceae, or any combination thereof.

In other embodiments, the green microalgae cell of the present invention can be a green microalgae cell from the genera of Dunaliella, Hafniomonas, Brachiomonas, Chlamydomonas, Chloromonas, Halosarcinochlamys, Lobochlamys, Oogamochlamys, Polytoma, Polytomella, Pseudocarteria, Vitreochlamys, Characiochloris, Golenkinia, Pyrobodys, Basichlamys, Tetrabaena, Basichlamys, Goniurn, Eudorina, Pandorina, Platydorina, Pleodorina, Volvox, Volvulina, Yamagishiella, Haematococcus, Stephcmosphaera, Asteromonas, Astrephomene, Phacotus, Pteromonas, Characiosiphon, Lobocharacium, Brachiomonas, Carteria, Chlainomonas, Lobomonas, Chlorella, Prototheca, Nannochloris, Pseudochlorella, Lobosphaera, Closteriopsis, Marvania, Viridiella, Auxenochlorella, Catena, Lobosphaeropsis, or Eremosphaera or any combination thereof.

In some embodiments, the green microalgae cell of present invention is not from the genus Chlamydomonas. In other embodiments of this invention, the green microalgae cell of present invention is not a Chlainydomonas species having a cell wall. In further embodiments, the green microalgae cell of present invention is not Chlamydomonas reinhardtii. In additional embodiments of this invention, the green microalgae cell of present invention can be a Chlamydomonas reinhardtii cell wall-deficient mutant.

In still other embodiments, green microalgae cell of the present invention can be Dunaliella salina, Dunaliella tertiolecta, Dunaliella primolecta, Dunaliella acidophilia, Dunaliella bardawil, Dunaliella lateralis, Dunaliella maritima, Dunaliella minuta, Dunaliella parva, Dunaliella peircei, Dunaliella polymorpha, Dunaliella pseudasalina, Dematiella quartolecta, Dunaliella viridis, Dunaliella sp. SPMA, or uncultured Dunaliella, or any combination thereof.

As described herein, the nucleic acid constructs of the present invention comprise a nucleotide sequence encoding an extremophile enzyme. The extremophile enzymes of the present invention can be any enzyme produced by an extremophile organism. In some embodiments of the present invention, the extremophile enzyme can be an oxidoreductase, a transferase, a hydrolase, a lyase, an isomerase, a ligase, or any combination thereof. In some embodiments of the invention, the oxidoreductase can be a cytochrome, a dehydrogenase, a dioxygenase, a laccase, a metalloreductase, a monoxygenase, or any combination thereof. In other embodiments of the present invention, the transferase can be an acyltransferase, an alkyltransferase, a carboxyltransferase, a fatty acyl synthase, a glycosyltransferase, a kinase, or any combination thereof. In some embodiments, the hydrolase can be an amylase, a cellulase, a glycosidase, a glucohydrolase, a glucanase, a lipase, a nuclease, a peptidase, a phosphatase, or any combination thereof. In further embodiments of this invention, the isomerase can be an epimerase, a foldase, a gyrase, a mutase, a racemase, a topoisomerase, or any combination thereof In additional embodiments of the present invention, the ligase can be an acyl synthetase, a carboxylase, a nucleic acid ligase, a peptide synthetase, or any combination thereof. In some embodiments, the lyase can be an acyl synthetase, a carboxylase, a nucleic acid ligase, a peptide synthetase, or any combination thereof. In some embodiments, the nucleotide sequences encoding the extremophile enzymes can be modified for codon usage bias, as described herein.

In some embodiments, the nucleic acid construct of the present invention comprises one or more heterologous nucleotide sequences encoding one or more extremophile enzymes (NSEE) (e.g., 1, 2, 3, 4, 5, 6, 7, 8, 9, 10, etc.). Thus, in some embodiments of the present invention, the nucleic acid construct comprises one, two, three, four, five six, seven, eight, nine, ten, or more, nucleotide sequences encoding an extremophile enzyme (e.g., NSEE1, NSEE2, NSEE3, NSEE4, NSEE5, NSEE6, NSEE7, NSEE8, NSEE9, NSEE10, and the like). In some embodiments, when the nucleic acid constructs of the present invention comprise more than one heterologous nucleotide sequence encoding an extremophile enzyme, the heterologous nucleotide sequences are each operably located sequentially in the nucleic acid construct, 3′ to the first promoter and 5′ to the first terminator. In some embodiments, the nucleic acid construct comprising more than one heterologous nucleotide sequence encoding an extremophile enzyme (NSEE) further comprises a ribosome binding site operably located 5′ of each of the NSEE in the nucleic acid construct. A non-limiting example of a nucleic acid construct comprising more than.one NSEE is a nucleic acid construct comprising in the following order from 5′ to 3′: (a) a left flanking sequence for homologous recombination (FS1); (b) a first promoter (P1); (c) a first enhancer sequence (EN1); (d) a ribosome binding site; (e) a heterologous nucleotide sequence encoding a first extremophile enzyme (NSEE1); (f) a ribosome binding site; (g) a heterologous nucleotide sequence encoding a second extremophile enzyme (NSEE2); (h) a ribosome binding site; (i) a heterologous nucleotide sequence encoding a third extremophile enzyme (NSEE3); (j) a ribosome binding site; (k) a heterologous nucleotide sequence encoding a fourth extremophile enzyme (NSEE4); (1) a first terminator (T1); (m) a right flanking sequence for homologous recombination (FS2); and wherein the one or more extremophile enzymes (NSEE) are modified for codon usage bias for the green microalgae cell; and the left and right flanking sequences for homologous recombination are nucleotide sequences that are homologous to the chloroplast genome of the green microalgae cell.

In some embodiments of the present invention, it may be useful to modify the lipids produced by the green microalgae prior to collection of the lipids from the green microalgae (i.e., in vivo). The term modify, modifying and/or modification (and grammatical variants thereof) as used herein with regard to lipids, means changing (e.g., increasing or decreasing) the amount of particular lipids of interest produced in a microalgae cell and/or the types of lipids produced in a microalgae cell as compared to the amount or types of lipids produced in a microalgae cell in which the lipids are not modified. Thus, for example, the quantity and/or types of lipids produced can be modified by transforming the green microalgae with one or more nucleotide sequences encoding enzymes for fatty acid synthesis. Thus, in one aspect of the invention, the chain lengths of the fatty acids produced can be changed or modified.

In further embodiments, the lipid production by the green microalgae can be modified in vivo by transforming the green microalgae with a nucleotide sequence encoding, for example, a lipase. The lipase produced by the transformed green microalgae can act on the lipids in the cytosol of the transformed green microalgae removing the fatty acid from the glycerol backbone, thereby allowing the free fatty acids to be excreted out of the cell and into the media where they are collected.

Accordingly, the present invention further provides nucleic acid constructs comprising a heterologous nucleotide sequence encoding one or more lipid modifying and/or lipid producing enzymes as described herein. A nucleic acid construct comprising a nucleotide sequence encoding one or more lipid modifying and/or lipid producing enzymes can be introduced into (transformed into) a green microalgae cell, thereby producing a stably transformed green microalgae cell expressing one or more heterologous lipid modifying enzymes. The heterologous nucleotide sequence encoding one or more lipid modifying and/or lipid producing enzymes can be introduced into the chloroplast and/or nuclear genome of the green microalgae cell. In some embodiments, the heterologous nucleotide sequence encoding one or more lipid modifying and/or lipid producing enzymes can be introduced into the green microalgae cell using, for example, a minichromosome vector.

Expression cassettes for transforming a green microalgae cell with a nucleotide sequence encoding one or more lipid modifying and/or lipid producing enzymes are constructed as described herein for nucleotide sequences encoding one or more extremophile enzymes or selection markers. Accordingly, in some embodiments, an expression cassette comprising a nucleotide sequence encoding one or more lipid modifying and/or lipid producing enzymes comprises a left flanking sequence-promoter-enhancer sequence-nucleotide sequence encoding one or more lipid modifying and/or lipid producing enzymes (NSLME, NSLPE)-terminator-right flanking sequence. The promoters, terminators and enhancers can be any promoter, terminator or enhancer functional in a green microalgae or cyanobacteria as described herein.

In some embodiments, a nucleic acid construct of the invention comprising a nucleotide sequence encoding one or more lipid modifying and/or lipid producing enzymes can further comprise a selection cassette as described herein for other nucleic acid constructs.

In further embodiments, the selection cassette and the nucleotide sequence encoding one or more lipid modifying and/or lipid producing enzymes are on separate expression cassettes. The separate expression cassettes are inserted into separate plasmids, which can be different or the same. The plasmids can then be separately propagated and mixed together before transformation of the microalgae, thereby co-transforming the two plasmids into the microalgae cells.

Non-limiting examples of lipid modifying and/or lipid producing enzymes useful with the present invention include acetyl-CoA carboxylase carboxyltransferase a, subunit, acetyl-CoA carboxylase carboxybiotin carrier protein, acetyl-CoA carboxylase biotin carboxylase, acetyl-CoA carboxylase carboxyltransferase β-subunit, malonyl CoA:ACP tranacylase, β-ketoacyl-ACP synthase III, β-ketoacyl-ACP synthase I, β-ketoacyl-ACP synthase II, β-ketoacyl-ACP reductase, β-hydroxyacyl-ACP dehydratase/isomerase, β-hydroxyacyl-ACP dehydratase, trans-2-enoyl-ACP reductase I, trans-2-enoyl-ACP reductase II, and trans-2-enoyl-ACP reductase III, a fatty acyl synthase, a lipase, a phospholipase, a saturase, a thioesterase, a desaturase, an enolase, and any combination thereof. In some embodiments of the present invention, the lipid modifying and/or lipid producing enzymes can be from an extremophile organism (i.e., an extremophile enzyme). In a further non-limiting example, the lipid modifying and/or lipid producing enzymes can be from a plant.

In some embodiments, similar to the nucleotide sequences encoding extremophile enzymes, the lipid modifying and/or lipid producing enzymes can be modified for codon usage bias using species specific codon usage tables. As described herein, when these nucleotide sequences are to be expressed in chloroplasts, the codon usage tables are generated based on a sequence analysis of the most highly expressed chloroplast genes for the green microalgae species of interest. When the nucleotide sequences are to be expressed in the nucleus, the codon usage tables are generated based on a sequence analysis of highly expressed nuclear genes for the green microalgae species of interest. The modifications for the nucleotide sequences for selection are determined by comparing the species specific codon usage table with the codons present in the native nucleotide sequences. In those embodiments in which each of codons in native nucleotide sequence for selection are sufficiently used, then no modifications are needed (e.g., a frequency of more than 30% for a codon used for a specific amino acid in that species would indicate no need for modification). In other embodiments, wherein up to 3 nucleotides have to be modified in the nucleotide sequence, site-directed mutagenesis can be used according to methods known in the art (Zheng et al. Nucleic Acids Res. 32:e115 (2004); Dammai, Meth. Mol. Biol 634:111-126 (2010); Davis and Vierstra. Plant Mol. Biol. 36(4): 521-528 (1998)). In still other embodiments, when more than three nucleotides changes are necessary, a synthetic nucleotide sequence can be generated using the same codon usage as the highly expressed chloroplast genes (or the highly expressed nuclear genes) that were used to develop the codon usage table.

Thus, the present invention is further envisioned to encompass nucleic acid constructs comprising one or more heterologous nucleotide sequences encoding one or more extremophile enzymes and/or one or more lipid modifying and/or lipid producing enzymes. In additional embodiments, the present invention provides nucleic acid constructs comprising one or more heterologous nucleotide sequences encoding one or more extremophile enzymes and/or one or more lipid modifying and/or lipid producing enzymes, wherein at least one lipid modifying and/or lipid producing enzyme is an extremophile enzyme.

In some embodiments of the present invention, in order to facilitate the purification of the recombinant enzymes from the green microalgae, the recombinant enzymes can be fused to N- or C-terminal affinity tags. Therefore, in some embodiments, a heterologous nucleotide sequence of the present invention encoding an extremophile enzyme and/or lipid modifying and/or lipid producing enzyme can further comprise a nucleotide sequence encoding a peptide tag that is fused to the 5’ or 3′ end of the nucleotide sequence encoding the extremophile enzyme and/or lipid modifying and/or lipid producing enzyme. Non-limiting examples of affinity tags that can be used with the present invention include a histidine (HIS) tag, a chitin-binding domain (CBD) tag, a glutathione-s-transferase (GST) tag, a strep II tag, a T7-tag, a FLAG® tag, an S-tag, a hemagglutinin (HA) epitope tag, a c-Myc tag, a DHFR tag, a calmodulin binding peptide (CBP) tag, a cellulose binding domain tag, a maltose-binding domain (MBD) tag, a Glutathione S Transferase (GST) tag, a Maltose Binding Protein (MBP) tag, and a T7 gene 10 tag. The recombinant extremozyme or other recombinant protein that is fused to a tag can then be purified using the appropriate affinity chromatography resin. Upon purification, the tag can be cleaved from the recombinant enzyme.

In addition, N- or C-terminal tags can be used with the present invention to stabilize the recombinant extremophile enzymes. Fusion of a recombinant enzyme with non-hydrophobic peptide tags can improve the solubility of the recombinant enzyme, thereby improving its stability and reducing its degradation. Thus, in some embodiments, a peptide tag is fused to the N- or C-terminus of an extremophile enzyme or other recombinant polypeptide of the present invention. Accordingly, in some embodiments, a heterologous nucleotide sequence of the present invention encoding an extremophile enzyme and/or lipid modifying and/or lipid producing enzyme can further comprise a nucleotide sequence encoding a peptide tag that is fused to the 5′ or 3′ end of the nucleotide sequence encoding the extremophile enzyme and/or lipid modifying and/or lipid producing enzyme. Examples of peptide tags which can be fused with the recombinant enzymes of this invention include, but are not limited to, a histidine (HIS) tag, a chitin-binding domain (CBD) tag, a glutathione-s-transferase (GST) tag, a strep II tag, a T7-tag, a FLAG® tag, an S-tag, a hemagglutinin (HA) epitope tag, a c-Myc tag, a DHFR tag, a calmodulin binding peptide (CBP) tag, a cellulose binding domain tag, a maltose-binding domain (MBD) tag, a Glutathione S Transferase (GST) tag, a Maltose Binding Protein (MBP) tag, a T7 gene 10 tag, a NusA tag, a thioredoxin tag, a small ubiquitin-like modifier (SUMO) tag, a ubiquitin tag, a GFP tag, and a mistic tag.

In additional embodiments, a heterologous nucleotide sequence is introduced into a green microalgae cell using microprojectile bombardment (ballistic) techniques as known in the art and as described herein. Thus, in some embodiments of this invention, a method for stably transforming a green microalgae cell with a heterologous nucleotide sequence is provided, the method comprising: propelling the heterologous nucleotide sequence at a green microalgae cell embedded in a gel at a velocity sufficient to pierce (e.g., penetrate, perforate puncture, and the like) the cell wall and/or cell membrane and/or chloroplast membrane and deposit the heterologous nucleotide sequence within the green microalgae cell or within a chloroplast of the microalgal cell; wherein the heterologous nucleotide sequence is incorporated into the green microalgae nuclear genome or the green microalgae chloroplast genome, thereby producing a stably transformed green microalgae cell, further wherein the heterologous nucleotide sequence is carried by a microprojectile and the heterologous nucleotide sequence is propelled at the green microalgae cell by propelling the microprojectile at the green microalgae cell. In some embodiments of this invention, the green microalgae cell may not have a cell wall. In that case, the heterologous nucleotide sequence is propelled at a green microalgae cell that is embedded in a gel at a velocity sufficient to pierce the cell membrane and/or chloroplast membrane and deposit the heterologous nucleotide sequence within the green microalgae cell or within a chloroplast of the microalgal cell.

In other embodiments, a method is provided for stably transforming a green microalgae cell with a heterologous nucleotide sequence, the method comprising: propelling the heterologous nucleotide sequence at a green microalgae cell embedded in a gel at a velocity sufficient to pierce the cell wall, cell membrane and chloroplast membrane and deposit the heterologous nucleotide sequence within a chloroplast of the green microalgal cell; wherein the heterologous nucleotide sequence is incorporated into the green microalgae chloroplast genome, thereby producing a stably transformed green microalgae cell, wherein the heterologous nucleotide sequence is carried by a microprojectile and the heterologous nucleotide sequence is propelled at the green microalgae cell by propelling the microprojectile at the green microalgae cell. In some embodiments of this invention, the green microalgae cell may not have a cell wall. In that case, the heterologous nucleotide sequence is propelled at a green microalgae cell that is embedded in a gel at a velocity sufficient to pierce the cell membrane and chloroplast membrane and deposit the heterologous nucleotide sequence within a chloroplast of the microalgal cell.

The green microalgae cell of the present invention can be any green microalgae cell as described herein. Thus, for example, in some embodiments of the present invention, the green microalgae cell can be a cell wall-less green microalgae cell.

While not wishing to be bound by any particular theory, it appears that embedding the green microalgae cell or cells in a gel may provide support needed by the cells when they are bombarded with the microprojectiles coated with the heterologous nucleic acid molecules of the invention, thereby keeping the cells from being destroyed by the impact of the projectiles. This novel approach overcomes the difficulties previously observed for the transformation of green microalgae, in particular, the transformation of the cell wall-less green microalgae.

Non-limiting examples of gelling agent that can be used to embed the cell or cells of the green microalgae include, but are not limited to, agar-agar, agarose, gellan gum (e.g., Gelrite®, Phytagel™), Difco Bacto® agar, Difco Noble® agar, Agar MBI-1®, Agar MBI-2®, carrageenan, phytoblend (Cassion Labs, North Logan, Utah), agarM, or any combination thereof In some embodiments of the present invention, the green microalgae cell or cells are embedded in a gel having a concentration of about 0.1% to about 2%. Thus, in some embodiments, the gel has a concentration of about 0.1%, about 0.2%, about 0.3%, about 0.4%, about 0.5%, about 0.6%, about 0.7% about 0.8%, about 0.9%, about 1.0%, about 1.1%, about 1.2%, about 1.3%, about 1.4%, about 1.5%, about 1.6%, about 1.7% about 1.8%, about 1.9%, about 2.0%, In other embodiments of the invention, the green microalgae cell or cells are embedded in a gel having a concentration of about 0.4%.

In some embodiments, the gelling agent that can be used to embed the green microalgae cell or cells is agar-agar agar) at a concentration of about 0.1% to about 2%. In other embodiments, the concentration of the agar-agar is 0.4%.

The microalgae cell or cells are mixed with the gel when the gel is liquid and warm but not hot (e.g., about 35° C. to about 70° C.). The gel comprising the microalgae cells is then allowed to cool and solidify, thereby embedding the cells in the gel. As used herein, the term “embedded” refers to the microalgae cell or cells being enclosed in the surrounding mass of the gel.

In general, the gel and cells are mixed in a proportion of 1:1. Therefore, the gel is prepared so that the initial concentration is about 2 times greater than the desired final concentration. Upon mixing the gel with the cells 1:1, the concentration of the gel will be halved and thus, will be at the desired final concentration. Thus, for example, if the desired final gel concentration is 0.4%, the gel is initially prepared at a concentration of 0.8%; mixing the gel with cells in a proportion of 1:1 results in a final gel concentration of 0.4%.

In some embodiments, the gel comprising the microalgae cell or cells is a thickness of about 2 mm to about 100 mm. Thus, in some embodiments, the gel thickness can be about 2 mm, about 3 mm, about 4 mm, about 5 mm, about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, about 20 mm, about 21 mm, about 22 mm, about 23 mm, about 24 mm, about 25 mm, about 26 mm, about 27 mm, about 28 mm, about 29 mm, about 30 mm, about 31 mm, about 32 mm, about 33 mm, about 34 mm, about 35 mm, about 36 mm, about 37 mm, about 38 mm, about 39 mm, about 40 mm, about 41 mm, about 42 mm, about 43 mm, about 44 mm, about 45 mm, about 46 mm, about 47 mm, about 48 mm, about 49 mm, about 50 mm, about 51 mm, about 52 mm, about 53 mm, about 54 mm, about 55 mm, about 56 mm, about 57 mm, about 58 mm, about 59 mm, about 60 mm, about 61 mm, about 62 mm, about 63 mm, about 64 mm, about 65 mm, about 66 mm, about 67 mm, about 68 mm, about 69 mm, about 70 mm, about 71 mm, about 72 mm, about 73 mm, about 74 mm, about 75 mm, about 76 mm, about 77 mm, about 78 mm, about 79 mm, about 80 mm, about 81 mm, about 82 mm, about 83 mm, about 84 mm, about 85 mm, about 86 mm, about 87 mm, about 88 mm, about 89 mm, about 90 mm, about 91 mm, about 92 mm, about 93 mm, about 94 mm, about 95 mm, about 96 mm, about 97 mm, about 98 mm, about 99 mm, about 100 mm, and the like, or any range therein.

In other embodiments, the microalgae cells can be pelleted, resuspended in a minimum amount of fresh media (e.g., about 10⁶ to 10⁹ cells/ml) and spread evenly in a layer on top of an agar plate (the thickness of the layer can be varied and the plate can be with or without antibiotic) (Boynton et al. (1988) Science 240: 1534-1538). In still other embodiments, the cells can be spread on top of a sterile filter disk placed atop a layer of sterile filter paper allowing the removal of media from the cells (Lerche and Hallmann (2009) BMC Biotechnology 9:64). After the desired cell dryness is achieved, the filter disk can be placed atop an agar plate (with or without antibiotic) and the transformation performed.

Generally, as known by those of skill in the art, “biolistics,” “biolistic bombardment” or “microprojectile bombardment” as a method of transformation involves propelling inert or biologically active particles at the green microalgae cells under conditions effective to penetrate the outer surface of the cell/cell membrane and/or chloroplast membrane and afford incorporation within the interior of the cell or chloroplast. See, e.g., U.S. Pat. Nos. 4,945,050; 5,036,006 and 5,100,792. This involves the use of high pressure and microparticles. The pressures and particle sizes used are optimized for the organism to be transformed. When inert particles are utilized, the vector or nucleic acid construct can be introduced into the cell by coating the particles with the vector (e.g., nucleic acid construct) containing the nucleotide sequence of interest (e.g., heterologous nucleotide sequence encoding an extremophile enzyme). Alternatively, in some embodiments, a cell or cells can be surrounded by a nucleic acid construct so that the nucleic acid construct is carried into the cell by the wake of the particle.

In some embodiments, microparticles for microprojectile bombardment can be comprised of gold or tungsten as is known in the art and the particles can be from about 50 mu to about 1600 nm in size. Accordingly, in representative embodiments, the particles can be about 50 nm, 60 nm, 70 nm, 80 nm, 90 nm, 100 nm, 110 nm, 120 nm, 130 nm, 140 nm, 150 nm, 160 mu, 170 nm, 180 nm, 190 nm, 200 nm, 210 nm, 220 nm, 230 nm, 240 nm, 250 nm, 260 nm, 270 nm, 280 nm, 290 nm, 300 nm, 310 nm, 320 nm, 330 nm, 340 nm, 350 nm, 360 nm, 370 nm, 380 nm, 390 nm, 400 nm, 410 nm, 420 nm, 430 nm, 440 nm, 450 nm, 460 nm, 470 nm, 480 nm, 490 nm, 500 nm, 510 mu, 520 nm, 530 nm, 540 nm, 550 nm, 560 nm, 570 nm, 580 nm, 590 nm, 600 nm, 610 nm, 620 nm, 630 nm, 640 nm, 650 nm, 660 nm, 670 nm, 680 nm, 690 nm, 700 nm, 710 nm, 720 nm, 730 nm, 740 nm, 750 nm, 760 nm, 770 nm, 780 nm, 790 nm, 800 nm, 810 nm, 820 nm, 830 nm, 840 nm, 850 nm, 860 nm, 870 nm, 880 nm, 890 nm, 900 nm, 910 nm, 920 nm, 930 mu, 940 nm, 950 nm, 960 nm, 970 nm, 980 nm, 990 nm, 1000 nm, 1050 nm, 1100 nm, 1150 nm, 1200 nm, 1250 nm, 1300 nm, 1350 nm, 1400 nm, 1450 nm, 1500 nm, 1550 nm, 1600 nm, and the like, or any range therein. Thus, in further embodiments, the particle size is in a range from about 50 nm to about 1000 nm, from about 100 nm to about 500 nm, from about 100 nm to about 250 nm, and the like. In some particular embodiments, the particle size is about 100 nm.

The heterologous nucleotide sequence used in the stable transformation of the green microalgae can be any nucleotide sequence of interest. Further, the nucleotide sequences of interest of this invention can be derived from any organism.

Thus, in some aspects of the invention, the heterologous nucleotide sequence used to transform the green microalgae cell can be any nucleic acid construct of the present invention as described herein. In other embodiments, the heterologous nucleotide sequence used to transform the green microalgae cell comprises a nucleotide sequence encoding one or more extremophile enzymes, as describe herein. In still other embodiments, the heterologous nucleotide sequence used to transform the green microalgae cell can comprise a nucleotide sequence encoding one or more lipid modifying and/or lipid producing enzymes, as describe herein. In further embodiments, the heterologous nucleotide sequence used to transform the green microalgae cell comprises a nucleotide sequence encoding pharmaceutical enzymes and/or vaccines. Non-limiting examples of heterologous nucleotide sequences useful with this invention include human prolidase, pathway for artemisinin production, tetanus toxin fragment C, canine parvo virus.

Additional methods of transformation that are well-known in the art for plant transformation can also be used for nuclear transformation of a green microalgae cell. Thus, for example, the present invention provides a method for stably transforming a green microalgae cell with a heterologous nucleotide sequence, the method comprising: subjecting green microalgae cells in the presence of the heterologous nucleotide sequence to osmotic shock, thereby creating pores in the green microalgae membrane; whereby the heterologous nucleotide sequence enters the cell and is incorporated into the nuclear or chloroplast genome, thereby producing a stably transformed green microalgae cell. Osmotic shock results when the osmolarity of the solution comprising the green microalgae cell or cells is altered by varying the amount of salt present in the solution. Osmolarity of a solution can also be altered using polyethylene glycol (PEG).

In other embodiments, the present invention provides a method for stably transforming a green microalgae cell with a heterologous nucleotide sequence, the method comprising: subjecting the cells in the presence of the nucleotide sequence to an electric shock, thereby creating pores in the green microalgae membrane, whereby the heterologous nucleotide sequence enters the cell and is incorporated into the nuclear or chloroplast genome, thereby producing a stably transformed green microalgae cell. Electric shock of the cells (electroporation) results in the creation of transient pores in the cell membranes, thereby allowing the nucleic acid constructs or heterologous nucleotide sequences to enter the cells. In some embodiments, for microalgae cells comprising cell walls, the cell walls can be removed prior to electroporation using enzymes as is well-known in the art (e.g., cellulase, autolysin, and the like) (Popper, et al. Annu. Rev. Plant Biol. 62:567-590 (2011); Harris, E. H. “The Chlamydomoncts Sourcebook: A Comprehensive Guide to Biology and Laboratory Use.” Academic Press, San Diego, Calif. (1989)).

In further embodiments, the present invention provides a method for stably transforming a green microalgae cell with a heterologous nucleotide sequence, the method comprising: vortexing the cells in the presence of the nucleotide sequence and a physical agent capable of creating pores (e.g., glass beads, silicon carbide whiskers, zirconia/silica beads, and the like), thereby disrupting the green microalgae membrane, whereby the heterologous nucleotide sequence enters the cell and is incorporated into the nuclear or chloroplast genome, thereby producing a stably transformed green microalgae cell.

As used herein, the term disrupt or disrupting as it pertains to the cell membrane refers to the formation of pores, transient pores or small holes in the membrane. These pores allow the vector, nucleic acid construct and/or heterologous nucleotide sequence to pass through the membrane and enter the cell, whereby the heterologous nucleotide sequence can be incorporated into the nuclear or chloroplast genome.

These and other methods for nuclear transformation are discussed generally in Boynton et al. Science 240:1534-1538 (1988); Brown et al. Mol. Cell. Biol. 11:2328-2332 (1991); Kindle K L Proc. Natl. Acad. Sci. USA 87:1228-1232 (1990); Dunahay T G Biotechniques 15:452-460 (1993); Kumar et al. Plant Science 166:731-73(2004); Sun et al. Mar. Biotechnol. 10:219-22(2008); Sun et al. Gene 377:140-14(2006); Lu et al. Sheng Wu Gong Cheng Xue Bao 25:520-5(2009); Jiang et al. Yi Chuan Xue Bao 32:424-43(2005); Tan et al. J. Microbiol. 43:361-3(2005); Feng et al. Mol. Biol. Reports 36:1433-143(2009); Miki et al. (“Procedures for Introducing Foreign DNA into Plants” in Methods in Plant Molecular Biology and Biotechnology); Glick, B. R. and Thompson, J. E., Eds. (CRC Press, Inc., Boca Raton, 1993, pages 67-88); Rakowoczy-Trojanowska (Cell. Mol. Biol. Lett. 7:849-858 (2002)); Horsch et al. (Science 227:1229 (1985); Ishida et al. (Nature Biotechnol. 14:745 750 (1996); Fraley et al. (Proc. Natl. Acad. Sci. 80: 4803 (1983); Broothaerts et al. (Nature 433:629-633 (2005)); Sanford et al. (Methods in Enzymology 217:483-509 (1993)); McCabe et al. (Plant Cell Tiss. Org. Cult. 33:227-236 (1993)) and Zhang et al. (Bio/Technology 9:996 (1991)).

In other embodiments of the invention, methods for increasing transformation efficiency can be used. Such methods include, but are not limited to, (1) synchronization of the green algae cultures prior to transformation; (2) performance of the transformation procedures at specific times in the cell cycle (e.g., if the culture conditions used a succession of light/dark cycles, transformation may be more efficient if the cells are transformed at the beginning or at the end of the light or dark cycle (Lapidot et al. Plant Physiology 129: 7-12 (2002))); and/or (3) applying an osmotic shock to the cells right before transformation.

The present invention further encompasses green microalgae and/or cyanobacteria cells in accordance with the embodiments of this invention. Thus, in some embodiments, the present invention provides a transformed green microalgae cell and/or a transformed cyanobacteria cell comprising a nucleic acid molecule, a nucleic acid construct, a nucleotide sequence, a promoter, and/or a composition of this invention.

In addition, the present invention further provides a method for producing one or more extremophile enzymes, the method comprising: (a) culturing a stably transformed green microalgae cell of the present invention that expresses one or more extremophile enzymes; and (b) collecting the one or more extremophile enzymes from the green microalgae culture of (a), thereby producing one or more extremophile enzymes.

In some additional aspects, the present invention provides a method for producing lipids and one or more extremophile enzymes in a green microalgae cell, the method comprising: (a) culturing a stably transformed microalgae cell of the present invention that expresses one or more extremophile enzymes, wherein the green microalgae cell further produces endogenous lipids; and (b) collecting both the endogenous lipids and the one or more extremophile enzymes from the green microalgae cell culture of (a), thereby producing lipids and extremophile enzymes from green microalgae.

In other aspects of this invention, a method is provided for producing modified lipids and one or more extremophile enzymes in a green microalgae cell, the method comprising: (a) culturing a stably transformed green microalgae cell of the present invention expressing one or more enzymes for modifying lipids and one or more extremophile enzymes, for a time sufficient for the one or more enzymes for modifying lipids to modify the lipids produced by the green microalgae cell; and (b) collecting both the modified lipids and the one or more extremophile enzymes from the green microalgae culture of (a). In some embodiments, the lipid modifying and/or lipid producing enzymes can be extremophile enzymes. In other embodiments, the lipid modifying and/or lipid producing enzymes are not extremophile enzymes. In some embodiments, the lipid modifying and/or lipid producing enzymes can be from plants. In still other embodiments, the lipid modifying enzymes can be a combination of lipid modifying and/or lipid producing extremophile enzymes and lipid modifying and/or lipid producing enzymes that are not from extremophile organisms.

In representative embodiments, “a time sufficient,” as used herein, refers to the time needed to reach mid- to late-log phase growth. As is known in the art, this time will be dependent on the algal species being grown and growth conditions provided.

In some aspects of this invention, the stably transformed green microalgae cell of step (a) comprises only one species or strain of green microalgae transformed as described herein and expressing at least one extremophile enzyme. In other embodiments, the stably transformed green microalgae cell of step (a) comprises cells from more than one green microalgae family, genus, species, and/or strain each of which is transformed as described herein and expressing at least one extremophile enzyme. Thus, in a non-limiting example, the present invention provides a method for producing one or more extremophile enzymes, the method comprising: (a) culturing a stably transformed green microalgae cell of the present invention that expresses one or more extremophile enzymes, wherein the stably transformed green microalgae cell comprises stably transformed green microalgae cells from the genus Dunaliella, the genus Volvulina and the genus Stephanosphaera; and (b) collecting the one or more extremophile enzymes from the green microalgae culture of (a).

In an additional non-limiting example, the present invention provides a method for producing one or more extremophile enzymes, the method comprising: (a) culturing a stably transformed green microalgae cell of the present invention that expresses one or more extremophile enzymes, wherein the stably transformed green microalgae cell comprises at least two species of Dunaliella (e.g., two, three, four, five, six, seven, eight, nine, ten, or more); and (b) collecting the one or more extremophile enzymes from the green microalgae culture of (a). In a further non-limiting example, the present invention provides a method for producing one or more extremophile enzymes, the method comprising: (a) culturing a stably transformed green microalgae cell of the present invention that expresses one or more extremophile enzymes, wherein the stably transformed green microalgae cell comprises at least two strains of Dunaliella salina (e.g., two, three, four, five, six, seven, eight, nine, ten, or more); and (b) collecting the one or more extremophile enzymes from the green microalgae culture of (a). Accordingly, any combination of green microalgae from any green microalgae family, genus, species, and/or strain can be used for the production of extremophile enzymes or extremophile enzymes and lipids for biofuel production as described herein.

The microalgae of the present invention can be cultured according to methods well known in the art. (See, e.g., The alga Dunaliella: biodiversity, physiology, genomics and biotechnology; A. Ben-Amotz, J. E. W. Polle, D. V. Subba Rao, Eds. Science Publishers (2009)). In general, the green microalgae can be cultured in a liquid culture comprising potassium nitrate, sodium chloride, potassium phosphate, bicarbonate and micronutrients. The green microalgae cultures can be grown under a light/dark regime or continuous light and supplemented with CO₂-enriched air. In other embodiments, the green microalgae can be grown or cultured on agar plates as described above. The present invention is envisioned to encompass large scale production of enzymes and lipid biofuel from the green microalgae. Thus, for industrial scale production of enzymes and/or lipids, the green microalgae can be grown in large scale in, for example, photobioreactors (indoors and/or outdoors) and/or in open systems including, but not limited to ponds, raceways, and the like or any combination thereof.

Collection of the lipids and the enzymes (proteins) can be performed using standard methods for collection and purification of proteins and lipids. Thus, for example, the green microalgae and/or cyanobacteria cells can be first concentrated by methods known in the art such as, for example, centrifugation, flotation, flocculation and any combination thereof (Williams and Laurens, Energy Environ. Sci. 3:554-590 (2010)). Following concentration, the cells of the green microalgae can be broken open (e.g., ruptured) using mechanical, enzymatic or chemical means (Id.). As a non-limiting example, changes in osmotic pressure can be used to rupture the algal cells. Thus, the green microalgae, which are cultured in media having an osmotic pressure higher (e.g., marine green algae) than water, can be resuspended in water causing the cells to rupture. Alternatively, after first being concentrated by centrifugation, the cells of green microalgae that are cultured in media having an osmotic pressure near or the same as water can be resuspended in a liquid having a higher osmotic pressure than water. The cells can then be once more concentrated by centrifugation and resuspended in water resulting in the rupturing of the cells.

Once the cells are opened, the neutral lipids (e.g., the lipids for biofuel production; triacylglycerides) float in the water and can be collected and purified using standard methods. The proteins (extremophile enzymes) can be purified using standard protein purification techniques. In some embodiments, the proteins are purified using affinity tag purification techniques. Thus, the proteins can be purified based on the particular affinity tag that is fused to the protein as described herein. In other embodiments of the present invention, the recombinant extremophile enzymes are purified using chromatography. In further embodiments of the present invention, purification and enrichment of the recombinant extremophile proteins can be achieved by using physical treatments that correspond to the type of extremophile enzyme produced. For instance, for enrichment of thermophilic/hyperthermophilic extremozymes, the protein extracts can be heat-treated at temperatures ranging from 60-100° C. (the temperature used depending on the heat stability of the extremozyme) in order to denature and remove heat labile host proteins from the extracts. To purify recombinant halophilic extremozymes, the extracts can be treated with salt solutions to salt out host protein, and to purify recombinant acidophilic and alkaliphilic extremozymes, extracts can be enriched for the recombinant proteins by treating the extracts with acid or base conditions.

### Cyanobacteria

The present invention further provides methods and compositions for the production of extremophile enzymes from cyanobacteria. In other embodiments, the present invention provides methods and compositions for the production of lipids and extremophile enzymes from cyanobacteria. In still other embodiments, the present invention provides methods and compositions for the production of modified lipids and extremophile enzymes from cyanobacteria.

In some embodiments of the present invention, the nucleic acid constructs for transformation of green microalgae chloroplasts as described herein can be used for the stable transformation of cyanobacteria, thereby producing extremophile enzymes in the stably transformed cyanobacteria. The nucleic acid constructs of the present invention for transformation of green microalgae chloroplasts can optionally be modified for cyanobacteria codon usage bias and selection of regulatory elements that are specific for cyanobacteria. In other embodiments, other nucleic acid constructs can be designed that comprise heterologous nucleotide sequences encoding for extremophile enzymes and used to stably transform cyanobacteria, thereby producing extremophile enzymes in the stably transformed cyanobacteria. Thus, for example, endogenous cyanobacteria plasmids as described by Xu et al. can be used for expression of the heterologous nucleotide sequences encoding for extremophile enzymes in cyanobacteria (Xu et al. Methods Mol. Biol. 684:273-293 (2011)).

Methods for transforming cyanobacteria are as described herein for green microalgae (e.g., biolistics, vortex/glass beads, electroporation, and the like). In other embodiments of the present invention, cyanobacteria can be transformed using natural transformation as described by Frigaard et al. (Methods Mol. Biol. 274:314-322 (2004)).

The extremophile enzymes of the present invention are as described herein.

In some embodiments of the present invention, the cyanobacteria can be any cyanobacteria. In other embodiments, the cyanobacteria of the present invention can be from the genus Synechococcus.

In addition, the present invention further provides a method for producing one or more extremophile enzymes, the method comprising: (a) culturing a stably transformed cyanobacteria cell of the present invention that expresses one or more extremophile enzymes; and (b) collecting the one or more extremophile enzymes from the cyanobacteria culture of (a).

In some further embodiments,.the present invention provides a method for producing lipids and one or more extremophile enzymes from cyanobacteria, the method comprising: (a) culturing a stably transformed cyanobacteria cell of the present invention that expresses one or more extremophile enzymes, wherein the cyanobacteria cell further produces endogenous lipids; and (b) collecting the endogenous lipids and the one or more extremophile enzymes from the cyanobacteria culture of (a), thereby producing lipids and extremophile enzymes from cyanobacteria.

In other aspects, the present invention provides a method for producing modified lipids and one or more extremophile enzymes from a cyanobacteria cell, the method comprising: (a) culturing a stably transformed cyanobacteria cell of the present invention expressing one or more enzymes for modifying lipids and one or more extremophile enzymes, for a time sufficient for the one or more enzymes for modifying lipids to modify the lipids produced by the cyanobacteria; and (b) collecting the modified lipids and the one or more extremophile enzymes from the cyanobacteria culture of (a).

Methods of culturing cyanobacteria are well known in the art and can be used for culturing the cyanobacteria of this invention. Thus, cyanobacteria can be cultured in the same way as green microalgae in liquid or solid medium. The medium contains macro-and micronutrients, can be supplemented with CO2 or bicarbonate at a pH appropriate for the specific Synechococcus species. The culturing can occur outdoors in large photobioreactors or indoors in a lab setting for smaller volumes or on solid or soft medium in petri dishes. (See, Ugwu et al. Biotechnol. Letters 27(2):75-78 (2003); Hemlata and Fatma. Bull. Environ. Contam. Toxicol. 83(4): 509-515 (2009); Tran et al. Biotechnol. Bioprocess Engineer. 15(2) 277-284 (2010)).

The lipids and the extremozymes (proteins) are collected using standard methods for collection and purification of proteins and lipids known in the art and as described herein for the lipids and extremophile enzymes produced by green microalgae.

### Definitions

As used herein, “a,” “an” or “the” can mean one or more than one. For example, a cell can mean a single cell or a multiplicity of cells.

As used herein, “and/or” refers to and encompasses any and all possible combinations of one or more of the associated listed items, as well as the lack of combinations when interpreted in the alternative (or).

Further, the term “about,” as used herein when referring to a measurable value such as an amount of a compound or agent, dose, time, temperature, activity, and the like, is meant to encompass variations of ±20%, ±10%, ±5%, ±1%, ±0.5%, or even ±0.1% of the specified amount.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.” Thus, the term “consists essentially of” (and grammatical variants), as applied to a polynucleotide sequence of this invention, means a polynucleotide that consists of both the recited sequence (e.g., SEQ ID NO) and a total of ten or less (e.g., 1, 2, 3, 4, 5, 6, 7, 8, 9, or 10) additional nucleotides on the 5′ and/or 3′ ends of the recited sequence such that the function of the polynucleotide is not materially altered. The total of ten or less additional nucleotides includes the total number of additional nucleotides on both ends added together.

Also as used herein, the terms “nucleic acid,” “nucleic acid molecule,” “nucleotide sequence” and “polynucleotide” refer to RNA or DNA that is linear or branched, single or double stranded, or a hybrid thereof. The term also encompasses RNA/DNA hybrids. When dsRNA is produced synthetically, less common bases, such as inosine, 5-methylcytosine, 6-methyladenine, hypoxanthine and others can also be used for antisense, dsRNA, and ribozyme pairing. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made.

As used herein, the term “nucleotide sequence” refers to a heteropolymer of nucleotides or the sequence of these nucleotides from the 5′ to 3′ end of a nucleic acid molecule and includes DNA or RNA molecules, including cDNA, a DNA fragment, genomic DNA, synthetic (e.g., chemically synthesized) DNA, plasmid DNA, mRNA, and anti-sense RNA, any of which can be single stranded or double stranded. The terms “nucleotide sequence” “nucleic acid,” “nucleic acid molecule,” “oligonucleotide” and “polynucleotide” are also used interchangeably herein to refer to a heteropolymer of nucleotides. Nucleic acid sequences provided herein are presented herein in the 5′ to 3′ direction, from left to right and are represented using the standard code for representing the nucleotide characters as set forth in the U.S. sequence rules, 37 CFR §§1.821-1.825 and the World Intellectual Property Organization (WIPO) Standard ST.25.

As used herein, the term “gene” refers to a nucleic acid molecule capable of being used to produce mRNA, antisense RNA, miRNA, and the like. Genes may or may not be capable of being used to produce a functional protein. Genes can include both coding and non-coding regions (e.g., introns, regulatory elements, promoters, enhancers, termination sequences and 5′ and 3′ untranslated regions). A gene may be “isolated” by which is meant a nucleic acid molecule that is substantially or essentially free from components normally found in association with the nucleic acid molecule in its natural state. Such components include other cellular material, culture medium from recombinant production, and/or various chemicals used in chemically synthesizing the nucleic acid molecule.

As used herein, the terms “fragment” or “portion” when used in reference to a nucleic acid molecule or nucleotide sequence will be understood to mean a nucleic acid molecule or nucleotide sequence of reduced length relative to a reference nucleic acid molecule or nucleotide sequence and comprising, consisting essentially of and/or consisting of a nucleotide sequence of contiguous nucleotides identical or almost identical (e.g., 80%, 81%, 82%, 83%, 84%, 85%, 86%, 87%, 88%, 89%, 90%, 91%, 92%, 93%, 94%, 95%, 96%, 97%, 98%, 99% identical) to the reference nucleic acid or nucleotide sequence. Such a nucleic acid fragment according to the invention may be, where appropriate, included in a larger polynucleotide of which it is a constituent.

An “isolated” nucleic acid molecule or nucleotide sequence or an “isolated” polypeptide is a nucleic acid molecule, nucleotide sequence or polypeptide that, by the hand of man, exists apart from its native environment and is therefore not a product of nature. An isolated nucleic acid molecule or isolated polypeptide may exist in a purified form or may exist in a non-native environment such as, for example, a recombinant host cell. Thus, for example, with respect to a polynucleotide, the term “isolated” means that it is separated from the chromosome and/or cell in which it naturally occurs and therefore is generally free of nucleotide sequences that flank the nucleic acid of interest in the genomic DNA of the organism from which the nucleic acid was derived (such as coding sequences present at the 5′ or 3′ ends). A polynucleotide is also isolated if it is separated from the chromosome and/or cell in which it naturally occurs and is then inserted into a genetic context, a chromosome and/or a cell in which it does not naturally occur. The recombinant nucleic acid molecules and nucleotide sequences of the invention can be considered to be “isolated” as defined above.

The nucleic acid molecule of this invention can include some additional bases or moieties that do not deleteriously or materially affect the basic structural and/or functional characteristics of the nucleic acid molecule.

Thus, an “isolated nucleic acid molecule” or “isolated nucleotide sequence” is a nucleic acid molecule or nucleotide sequence that is not immediately contiguous with nucleotide sequences with which it is immediately contiguous (one on the 5′ end and one on the 3′ end) in the naturally occurring genome of the organism from which it is derived. Accordingly, in one embodiment, an isolated nucleic acid includes some or all of the 5′ non-coding (e.g., promoter) sequences that are immediately contiguous to a coding sequence. The term therefore includes, for example, a recombinant nucleic acid that is incorporated into a vector, into an autonomously replicating plasmid or virus, or into the genomic DNA of a prokaryote or eukaryote, or which exists as a separate molecule (e.g., a cDNA or a genomic DNA fragment produced by PCR or restriction endonuclease treatment), independent of other sequences. It also includes a recombinant nucleic acid that is part of a hybrid nucleic acid molecule encoding an additional polypeptide or peptide sequence.

The term “isolated” can further refer to a nucleic acid molecule, nucleotide sequence, polypeptide, peptide or fragment that is substantially free of cellular material, viral material, and/or culture medium (e.g., when produced by recombinant DNA techniques), or chemical precursors or other chemicals (e.g., when chemically synthesized). Moreover, an “isolated fragment” is a fragment of a nucleic acid molecule, nucleotide sequence or polypeptide that is not naturally occurring as a fragment and would not be found as such in the natural state. “Isolated” does not mean that the preparation is technically pure (homogeneous), but it is sufficiently pure to provide the polypeptide or nucleic acid in a form in which it can be used for the intended purpose.

In representative embodiments of the invention, an “isolated” nucleic acid molecule, nucleotide sequence, and/or polypeptide is at least about 5%, 10%, 15%, 20%, 25%, 30%, 40%, 50%, 60%, 70%, 75%, 80%, 85%, 90%, 95%, 96% 97%, 98%, 99% pure (w/w) or more. In other embodiments, an “isolated” nucleic acid, nucleotide sequence, and/or polypeptide indicates that at least about a 5-fold, 10-fold, 25-fold, 100-fold, 1000-fold, 10,000-fold, 100,000-fold or more enrichment of the nucleic acid (w/w) is achieved as compared with the starting material.

As used herein, “complementary” polynucleotides are those that are capable of hybridizing via base pairing according to the standard Watson-Crick complementarity rules. Specifically, purines will base pair with pyrimidines to form a combination of guanine paired with cytosine (G:C) and adenine paired with either thymine (A:T) in the case of DNA, or adenine paired with uracil (A:U) in the case of RNA. For example, the sequence “A-G-T” binds to the complementary sequence “T-C-A.” It is understood that two polynucleotides may hybridize to each other even if they are not completely or fully complementary to each other, provided that each has at least one region that is substantially complementary to the other.

The terms “complementary” or “complementarity,” as used herein, refer to the natural binding of polynucleotides under permissive salt and temperature conditions by base-pairing. Complementarity between two single-stranded molecules may be “partial,” in which only some of the nucleotides bind, or it may be complete when total complementarity exists between the single stranded molecules either along the full length of the molecules or along a portion or region of the single stranded molecules. The degree of complementarity between nucleic acid strands has significant effects on the efficiency and strength of hybridization between nucleic acid strands.

As used herein, the terms “substantially complementary” or “partially complementary” mean that two nucleic acid sequences are complementary at least at about 50%, 60%, 70%, 80% or 90% of their nucleotides. In some embodiments, the two nucleic acid sequences can be complementary at least at about 85%, 90%, 95%, 96%, 97%, 98%, 99% or more of their nucleotides. The terms “substantially complementary” and “partially complementary” can also mean that two nucleic acid sequences can hybridize under high stringency conditions and such conditions are well known in the art.

As used herein, “heterologous” refers to a nucleic acid molecule or nucleotide sequence that either originates from another species or is from the same species or organism but is modified from either its original form or the form primarily expressed in the cell. Thus, a nucleotide sequence derived from an organism or species different from that of the cell into which the nucleotide sequence is introduced, is heterologous with respect to that cell and the cell's descendants. In addition, a heterologous nucleotide sequence includes a nucleotide sequence derived from and inserted into the same natural, original cell type, but which is present in a non-natural state, e.g. present in a different copy number, and/or under the control of different regulatory sequences than that found in the native state of the nucleic acid molecule.

As used herein, the terms “transformed” and “transgenic” refer to any green microalgae cell, and/or cyanobacteria cell that contains all or part of at least one recombinant (e.g, heterologous) polynucleotide. In some embodiments, all or part of the recombinant polynucleotide is stably integrated into a chromosome or stable extra-chromosomal element, so that it is passed on to successive generations. For the purposes of the invention, the term “recombinant polynucleotide” refers to a polynucleotide that has been altered, rearranged, or modified by genetic engineering. Examples include any cloned polynucleotide, or polynucleotides, that are linked or joined to heterologous sequences. The term “recombinant” does not refer to alterations of polynucleotides that result from naturally occurring events, such as spontaneous mutations, or from non-spontaneous mutagenesis followed by selective breeding.

The term “transgene” as used herein, refers to any nucleotide sequence used in the transformation of a microalgae, cyanobacteria, bacteria, plant, animal, or other organism. Thus, a transgene can be a coding sequence, a non-coding sequence, a cDNA, a gene or fragment or portion thereof, a genomic sequence, a regulatory element and the like. A “transgenic” organism, such as a transgenic green microalgae, transgenic plant, transgenic microorganism, or transgenic animal, is an organism into which a transgene has been delivered or introduced and the transgene can be expressed in the transgenic organism to produce a product, the presence of which can impart an effect and/or a phenotype in the organism.

Different nucleotide sequences or polypeptide sequences having homology are referred to herein as “homologues.” The term homologue includes homologous sequences from the same and other species and orthologous sequences from the same and other species. “Homology” refers to the level of similarity between two or more nucleotide sequences and/or amino acid sequences in terms of percent of positional identity (i.e., sequence similarity or identity). Homology also refers to the concept of similar functional properties among different nucleic acids, amino acids, and/or proteins.

As used herein “sequence identity” refers to the extent to which two optimally aligned polynucleotide or polypeptide sequences are invariant throughout a window of alignment of components, e.g., nucleotides or amino acids. “Identity” can be readily calculated by known methods including, but not limited to, those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991).

As used herein, the term “substantially identical” or “corresponding to” means that two nucleotide sequences have at least 50%, 60%, 70%, 75%, 80%, 85%, 90% or 95% sequence identity. In some embodiments, the two nucleotide sequences can have at least 85%, 90%, 95%, 96%, 97%, 98%, 99% or 100% sequence identity.

An “identity fraction” for aligned segments of a test sequence and a reference sequence is the number of identical components which are shared by the two aligned sequences divided by the total number of components in the reference sequence segment, i.e., the entire reference sequence or a smaller defined part of the reference sequence. Percent sequence identity is represented as the identity fraction multiplied by 100. As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned (with appropriate nucleotide insertions, deletions, or gaps totaling less than 20 percent of the reference sequence over the window of comparison). In some embodiments, “percent identity” can refer to the percentage of identical amino acids in an amino acid sequence.

Optimal alignment of sequences for aligning a comparison window is well known to those skilled in the art and may be conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCG® Wisconsin Package® (Accelrys Inc., Burlington, Mass.). The comparison of one or more polynucleotide sequences may be to a full-length polynucleotide sequence or a portion thereof, or to a longer polynucleotide sequence. For purposes of this invention “percent identity” may also be determined using BLASTX version 2.0 for translated nucleotide sequences and BLASTN version 2.0 for polynucleotide sequences.

The percent of sequence identity can be determined using the “Best Fit” or “Gap” program of the Sequence Analysis Software Package™ (Version 10; Genetics Computer Group, Inc., Madison, Wis.). “Gap” utilizes the algorithm of Needleman and Wunsch (Needleman and Wunsch, J Mol. Biol. 48:443-453, 1970) to find the alignment of two sequences that maximizes the number of matches and minimizes the number of gaps. “BestFit” performs an optimal alignment of the best segment of similarity between two sequences and inserts gaps to maximize the number of matches using the local homology algorithm of Smith and Waterman (Smith and Waterman, Adv. Appl. Math., 2:482-489, 1981, Smith et al., Nucleic Acids Res. 11:2205-2220, 1983).

Useful methods for determining sequence identity are also disclosed in Guide to Huge Computers (Martin J. Bishop, ed., Academic Press, San Diego (1994)), and Carillo et al. (Applied Math 48:1073(1988)). More particularly, preferred computer programs for determining sequence identity include but are not limited to the Basic Local Alignment Search Tool (BLAST) programs which are publicly available from National Center Biotechnology Information (NCBI) at the National Library of Medicine, National Institute of Health, Bethesda, Md. 20894; see BLAST Manual, Altschul et al., NCBI, NLM, NIH; (Altschul et al., J. Mol. Biol. 215:403-410 (1990)); version 2.0 or higher of BLAST programs allows the introduction of gaps (deletions and insertions) into alignments; for peptide sequence BLASTX can be used to determine sequence identity; and for polynucleotide sequence BLASTN can be used to determine sequence identity.

Accordingly, the present invention further provides nucleotide sequences having significant sequence identity to the nucleotide sequences of the present invention. Significant sequence similarity or identity means at least 70%, 75%, 80%, 81%, 82%, 83%, 84%, 85%, 86%, 87%, 88%, 89%, 90%, 91%, 92%, 93%, 94%, 95%, 96%, 97%, 98%, 99% and/or 100% similarity or identity with another nucleotide sequence.

Where more than one nucleic acid molecule is to be introduced into the green microalgae cell and/or chloroplast and/or the cyanobacteria cell, these nucleic acid molecules can be assembled as part of a single polynucleotide or nucleic acid construct, or as separate polynucleotide or nucleic acid constructs, and can be located on the same or different nucleic acid constructs. “Introducing” in the context of a green microalgae cell and/or chloroplast and/or the cyanobacteria cell means contacting a nucleic acid molecule with the green microalgae cell and/or chloroplast and/or the cyanobacteria cell in such a manner that the nucleic acid molecule gains access to the interior of the green microalgae cell and/or chloroplast and/or the cyanobacteria cell. Accordingly, these polynucleotides can be introduced into green microalgae cells and/or chloroplasts and/or the cyanobacteria cells in a single transformation event, in separate transformation events, or, e.g., as part of a breeding protocol. Thus, the term “transformation” as used herein refers to the introduction of a heterologous nucleic acid into a cell and/or chloroplast. Transformation of a green microalgae cell and/or chloroplast and/or the cyanobacteria cell may be stable or transient.

“Transient transformation” in the context of a polynucleotide means that a polynucleotide is introduced into the cell and does not integrate into the genome of the cell or chloroplast.

By “stably introducing” or “stably introduced” in the context of a polynucleotide introduced into a cell, it is intended that the introduced polynucleotide is stably incorporated into the nuclear and/or chloroplast genome of the cell, and thus the cell is stably transformed with the polynucleotide.

“Stable transformation” or “stably transformed” as used herein means that a nucleic acid molecule is introduced into a cell and integrates into the nuclear and/or chloroplast genome of the cell. As such, the integrated nucleic acid molecule is capable of being inherited by the progeny thereof, more particularly, by the progeny of multiple successive generations. “Genome” as used herein includes the nuclear and/or plastid genome, and therefore includes integration of the nucleic acid into, for example, the chloroplast genome. Stable transformation as used herein can also refer to a nucleic acid molecule that is maintained extrachromasomally, for example, as a minichromosome.

Transient transformation may be detected by, for example, an enzyme-linked immunosorbent assay (ELISA) or Western blot, which can detect the presence of a peptide or polypeptide encoded by one or more nucleic acid molecules introduced into an organism. Stable transformation of a cell can be detected by, for example, a Southern blot hybridization assay of genomic DNA of the cell with nucleic acid sequences which specifically hybridize with a nucleotide sequence of a nucleic acid molecule introduced into an organism (e.g., a plant). Stable transformation of a cell can be detected by, for example, a Northern blot hybridization assay of RNA of the cell with nucleic acid sequences which specifically hybridize with a nucleotide sequence of a nucleic acid molecule introduced into a plant or other organism. Stable transformation of a cell can also be detected by, e.g., a polymerase chain reaction (PCR) or other amplification reaction as are well known in the art, employing specific primer sequences that hybridize with target sequence(s) of a nucleic acid molecule, resulting in amplification of the target sequence(s), which can be detected according to standard methods Transformation can also be detected by direct sequencing and/or hybridization protocols well known in the art.

In addition to the promoters operably linked to the nucleic acid constructs, and/or nucleotide sequences of the present invention (e.g., nucleic acid constructs and/or nucleotide sequences encoding extremophile enzymes) described above, the expression cassette also can include other regulatory sequences. As used herein, “regulatory sequences” means nucleotide sequences located upstream (5′ non-coding sequences), within or downstream (3′ non-coding sequences) of a coding sequence, and which influence the transcription, RNA processing or stability, or translation of the associated coding sequence. Regulatory sequences include, but are not limited to, enhancers, introns, translation leader sequences and polyadenylation signal sequences as described herein.

A signal sequence can be operably linked to a nucleic acid molecule of the present invention to direct the nucleic acid molecule into a cellular compartment. In this manner, the expression cassette will comprise a nucleic acid molecule of the present invention operably linked to a nucleotide sequence for the signal sequence. The signal sequence may be operably linked at the N- or C-terminus of the nucleic acid molecule. Thus in some embodiments of the invention, species and strain specific target or retention signals can be used to targeting the nuclear expressed recombinant proteins, for example, to the chloroplast, into the secretory pathway for excretion into the medium, and/or for retention in the endoplasmic reticulum.

As used herein, “operably linked” means that elements of a nucleic acid construct such as an expression cassette are configured so as to perform their usual function. Thus, regulatory or control sequences (e.g., promoters) operably linked to a nucleotide sequence of interest are capable of effecting expression of the nucleotide sequence of interest. Further, control sequences can be regulated by regulatory sequences.

The control sequences need not be contiguous with the nucleotide sequence of interest, so long as they function to direct the expression thereof. Thus, for example, intervening untranslated, yet transcribed, sequences can be present between a promoter and a coding sequence, and the promoter sequence can still be considered “operably linked” to the coding sequence.

The following examples are not intended to be a detailed catalog of all the different ways in which the present invention may be implemented or of all the features that may be added to the present invention. Persons skilled in the art will appreciate that numerous variations and additions to the various embodiments may be made without departing from the present invention. Hence, the following descriptions are intended to illustrate some particular embodiments of the invention, and not to exhaustively specify all permutations, combinations and variations thereof.

### EXAMPLES

The invention described herein provides a novel method for expression of extremozymes as co-products in biofuel-producing green microalgae and cyanobacteria. Species specific synthetic genes are generated for insertion into species specific transformation cassettes. These synthetic DNA constructs can be transformed into the respective organisms by methodologies as described herein. Transgenic cells are selected and grown for expression of one or more extremozymes. Confirmation of the transformation and production of the one or more extremophile enzymes can be carried out by, for example, gene-specific PCR and protein-specific Western blot analysis.

### Example 1

### Codon Optimization of Transgenes

Codon usage bias is a species specific deviation from the uniform codon usage in the coding regions and is mainly based on tRNA copy number and genomic % GC. Codon usage bias of individual nucleotide sequences can correlate with expression levels. For expression of recombinant protein in green microalgae, codon-usage bias has been analyzed for Dunaliella salina homologs of Chlamydomonas reinhardtii highly expressed chloroplast and nuclear genes and reference tables generated for the respective codon preferences using OPTIMIZER as described in Puigbo et al. (Nucl. Acids Res. 35:gkm219 (2007)). Table 1 shows the codon usage bias for Dunaliella salina.

TABLE 1
Dunaliella salina codon usage bias for nuclear
and chloroplast nucleotide sequences.

| | | | Bias normalized to | |
|---|---|---|---|---|
| | | | Amino Acid Max | |
| | | | Highly Expressed | Chloroplastic |
| Symbol | Amino acids | Codons | Nuclear Genes | Genes |
| A | Alanine | GCT | 1 | 1 |
| A | Alanine | GCC | 0.53763 | 0.1223 |
| A | Alanine | GCA | 0.97849 | 0.58273 |
| A | Alanine | GCG | 0.26344 | 0.11366 |
| C | Cysteine | TGT | 0.45098 | 1 |
| C | Cysteine | TGC | 1 | 0.09316 |
| D | Aspartic acid | GAT | 0.70114 | 1 |
| D | Aspartic acid | GAC | 1 | 0.26074 |
| E | Glutamic acid | GAA | 0.52331 | 1 |
| E | Glutamic acid | GAG | 1 | 0.1002 |
| F | Phenyl alanine | TTT | 0.53503 | 1 |
| F | Phenyl alanine | TTC | 1 | 0.41737 |
| G | Glycine | GGT | 0.9424 | 1 |
| G | Glycine | GGC | 1 | 0.07296 |
| G | Glycine | GGA | 0.27225 | 0.33476 |
| G | Glycine | GGG | 0.20942 | 0.08047 |
| H | Histidine | CAT | 0.30645 | 1 |
| H | Histidine | CAC | 1 | 0.47985 |
| I | Isoleucine | ATT | 0.74166 | 1 |
| I | Isoleucine | ATC | 1 | 0.16077 |
| I | Isoleucine | ATA | 0.025 | 0.23674 |
| K | Lysine | AAA | 0.30288 | 1 |
| K | Lysine | AAG | 1 | 0.07328 |
| L | Leucine | TTA | 0.34 | 1 |
| L | Leucine | TTG | 0.21 | 0.06223 |
| L | Leucine | CTT | 0.185 | 0.19408 |
| L | Leucine | CTC | 0.195 | 0.00616 |
| L | Leucine | CTA | 0.1 | 0.09981 |
| L | Leucine | CTG | 1 | 0.01663 |
| M | Methionine | ATG | 1 | 1 |
| | (Start) | | | |
| N | Asparagine | AAT | 0.23225 | 1 |
| N | Asparagine | AAC | 1 | 0.32036 |
| P | Proline | CCT | 0.83495 | 1 |
| P | Proline | CCC | 1 | 0.08616 |
| P | Proline | CCA | 0.67961 | 0.88208 |
| P | Proline | CCG | 0.1165 | 0.15878 |
| Q | Glutamine | CAA | 0.48251 | 1 |
| Q | Glutamine | CAG | 1 | 0.07954 |
| R | Arginine | CGT | 0.93975 | 1 |
| R | Arginine | CGC | 1 | 0.10198 |
| R | Arginine | CGA | 0.16867 | 0.21104 |
| R | Arginine | CGG | 0.2771 | 0.00708 |
| R | Arginine | AGA | 0.15662 | 0.33852 |
| R | Arginine | AGG | 0.42168 | 0.01138 |
| S | Serine | TCT | 0.65432 | 1 |
| S | Serine | TCC | 0.88888 | 0.09214 |
| S | Serine | TCA | 0.72839 | 0.87915 |
| S | Serine | TCG | 0.11111 | 0.1858 |
| S | Serine | AGT | 0.30864 | 0.56797 |
| S | Serine | AGC | 1 | 0.09063 |
| T | Threonine | ACT | 0.76923 | 0.92096 |
| T | Threonine | ACC | 1 | 0.1 |
| T | Threonine | ACA | 0.58119 | 1 |
| T | Threonine | ACG | 0.1282 | 0.13064 |
| V | Valine | GTT | 0.375 | 1 |
| V | Valine | GTC | 0.34523 | 0.059 |
| V | Valine | GTA | 0.34523 | 0.75931 |
| V | Valine | GTG | 1 | 0.08074 |
| W | Tryptophan | TGG | 1 | 1 |
| V | Tyrosine | TAT | 0.43137 | 1 |
| Y | Tyrosine | TAC | 1 | 0.26247 |
| | STOP | TAA | 1 | 1 |
| | STOP | TAG | 0.22222 | 0.04918 |
| | STOP | TGA | 0.11111 | 0 |

The codon usage bias table can be used as described herein to generate synthetic genes for the recombinant proteins based on the extremophile DNA sequences with optimal codon composition for high expression in Dunaliella salina. Such reference tables can be generated for other green microalgae or for cyanobacteria.

Thus, codon-usage tables can be generated from any organism with sequence information from a sufficiently large set of chloroplast or nuclear genes (20 or more) that are highly expressed. This information is generally available at the National Center for Biotechnology Information (www.ncbi.nlm.nih.gov/) or other sources. The frequency of codons present in these highly expressed genes is normalized and compared to the equivalent table for the specific green microalgae or cyanobacteria and a table such as provided above for Dunaliella salina is generated.

### Vector Construction

The vectors developed for both nuclear and chloroplast transformation methods comprise a plasmid vector that allows for the propagation of the vector in Escherichia coli. The plasmid vector further comprises a transformation/expression cassette (e.g., nucleic acid construct). The transformation/expression cassette includes all elements necessary for integration and expression of the heterologous nucleotide sequence encoding one or more extremophile enzymes into the green microalgae or cyanobacteria. Non-limiting examples of expression cassettes of this invention are provided below.

### Example 2

### Expression Cassettes (Nucleic Acid Constructs) for Chloroplast and Nuclear Transformation

The generation of expression cassettes for transformation is based on an adaptation of the overlap PCR technique (See, Horton. Molecular Biotechnology 3: 93-99 (1995); and Wurch et al, Biotechnology Techniques 12: 653-657. (1998)). Different fragments are first amplified with overlapping sequences to create full length product by combining overlapping fragments.

The nucleotide sequences to be expressed by the green microalgae are modified for codon usage using the codon usage tables generated for the specific green microalgae or cyanobacteria to be transformed (both nuclear and chloroplast genome codon usage) (see e.g., Table 1). Accordingly, the extremozyme nucleotide sequences are synthesized for optimal chloroplast or nuclear codon composition. In addition, the nucleotide sequences conferring traits that allow for the selection of transformed green microalgae cells and the nucleotide sequences encoding other heterologous polypeptides of this invention can be synthesized for optimal chloroplast or nuclear codon composition as described herein.

Expression of Prolidase from the Extremophile Pyrococcus horikoshii in the Green Microalgae Dunaliella salina

Prolidases (EC 3.4,13.9) have been isolated from Archaea (Ghosh et al. J. Bacteriol. 180:4781-4789 (1998)), Bacteria (Suga et al. Biosci Biotechnol Biochem 59:2087-2090 (1995); Fujii et al. Biosci Biotechnol Biochem 60:1118-1122 (1996); Fernandez-Espla et al. Appl Environ Microbiol 63:314-316 (1997); Kabashima et al. Biochim Biophys Acta 1429:516-520 (1999); Park et al. 429:224-230 (2004)), and Eukaryotes (Sjostrom et al. Biochim Biophys Acta 327:457-470 (1973); Browne et al. J Biol Chem 258:6147-6154 (1983); Endo et al. J Biol Chem 264:4476-4481 (1989); Myara et al. Int J Biochem 26:207-214 (1994); Jalving et al. Mol Genet Genomics 267:218-222 (2002)). In archaea and bacteria prolidase aids in protein degradation and is responsible for recycling proline (Grunden et al. Methods Enzymol 330:433-445 (2001)). In humans, it is known that prolidase is involved in the final stage of the degradation of endogenous and dietary protein and is important in collagen catabolism (Endo et al. J Biol Chem 264:4476-4481 (1989); Forlino et al. Hum Genet 111:314-322 (2002))

Recombinant prolidase has use in several biotechnological applications (Theriot et al. Adv Appl Microbiol 68: 99-132 (2009)). In addition to reducing the bitterness of cheese (Bockelmann, International Dairy Journal 5:977-994 (1995)), prolidases also can degrade organophosphorus (OP) nerve agents, which act by inhibiting acetylcholinesterase (AChE), leading to a buildup of acetylcholine in the body and causing convulsions, respiratory problems, coma and death. Currently, thermolabile mesophilic Alteromonas prolidases are used in a foam formulation for biodecontamination of OP nerve agents, but because of the harsh conditions and broad temperature ranges in which the enzymes must operate, there has been interest in isolating and characterizing thermostable prolidases for use in bio-decontamination applications (Joint Science and Technology Office for Chemical and Biological Defense FY10/11: New Initiatives, 2008). Therefore, expression of a thermoactive P. horikoshii Ph1pro1 enzyme may be a value-added co-product in Dunaliella for use in biodecontamination.

The present inventors have successfully cloned Pyrococcus horikoshii prolidase encoded by ORF PH0974 (Ph1pro1) into the E. coli T7-RNA polymerase based expression vector pET-21b (Theriot et al. Appl Microbiol Biotechnol 86:177-188 (2010)). A synthetic version of the ORF PH0974 gene can be prepared for generation of the green microalgae and cyanobacteria expression constructs.

### Nucleic Acid Construct for Chloroplast Transformation.

An example of a nucleic acid construct comprising a heterologous nucleotide sequence encoding Pyrococcus horikoshii prolidase (that has been modified for D. salina codon usage) for chloroplast transformation of Dunaliella salina is provided by the nucleotide sequence of SEQ ID NO:1. SEQ ID NO:1 comprises a nucleic acid construct comprising a left flanking sequence of about 1000 base pairs (LB200 L-Flank: nucleotides (nt) 1-1039), a promoter from the atpA gene (LB200 PatpA: nt 1040-1592), a first ribosome binding site (RBS1: nt 1593-1607), a nucleotide sequence conferring chloramphenicol resistance for selection (ct-cat: nt 1608-2264), a second ribosome binding site (RBS2: nt 2265-2279), a heterologous nucleotide sequence encoding Pyrococcus horikoshii prolidase modified for D. salina codon usage (ct-prolidase: nt 2280-3347), a nucleotide sequence encoding an affinity tag (His-Tag: nt 3348-3365), a terminator from the psbA gene (LB200 TpsbA: nt 3366-3753) and a right flanking sequence of about 900 base pairs (LB200 R-Flank: nt 3754-4637).

### Nucleic Acid Construct for Nuclear Transformation.

An example of a nucleic acid construct comprising a heterologous nucleotide sequence encoding Pyrococcus horikoshii prolidase (that has been modified for D. salina codon usage) for nuclear transformation of Dunaliella salina is provided by the nucleotide sequence of SEQ ID NO:2. SEQ ID NO:2 comprises a promoter from the RbcS1 gene (P-RbcS1: nt 1-180), a heterologous nucleotide sequence encoding Pyrococcus horikoshii prolidase modified for D. salina codon usage (nu-prolidase: nt 181-1248), a nucleotide sequence encoding a linker (GS: nt 1249-1254), a nucleotide sequence conferring resistance to bleomycin for use in selection (ble: nt 1255-1626), a nucleotide sequence encoding an affinity tag (His-Tag: nt 1627-1644), and a terminator from the RbcS1 gene (T-RbcS1: nt 1645-1980).

Expression of Aminoacylase from the Extremophile Pyrococcus horikoshii in the Green Microalgae Dunaliella salina.

Aminoacylase (EC 3.5.1.14) is one of the most important enzymes used in industrial biotechnology because it can enantioselectively liberate L-amino acids from a corresponding N-acyl-amino acid racemate (Birnbaum et al. J Biol Chem 194:455-470 (1952)). Aminoacylases are used industrially for large-scale resolution of L-alanine, L-methionine, L-phenylalanine, and L-valine in excess of 100 tons per year (Tewari, Appl Biochem Biotechnol 23:187-203 (1990); Sakanyan et al. Appl Environ Microbiol 59:3878-3888 (1993); Bommarius et al. Journal of Molecular Catalysis B: Enzymatic 5:1-11 (1998); Toogood et al. Extremophiles 6:111-122 (2002); Koreishi et al. Biosci Biotechnol Biochem 69:1914-1922 (2005)). A number of recombinant thermoactive aminoacylases have been produced in Escherichia coli for use in industry (Story et al. J Bacteriol 183:4259-4268 (2001); Toogood et al. Extremophiles 6:111-122 (2002); Tanimoto et al. FEBS J275: 1140-1149 (2008)). One of these thermoactive aminoacylases, the aminocylase (phoACY) encoded by ORF PH0722 in Pyrococcus horikioshii is very thermostable; has maximum activity at 90° C. and is able to efficiently release amino acids from the substrates N-acetyl-L-methionine, N-acetyl-L-glutamine, and N-acetyl-L-leucine (Tanimoto et al. FEBS J275: 1140-1149 (2008)). The P. horikoshii aminoacylase will be expressed in Dunaliella as a value added co-product to lipid biofuel production.

A synthetic version of the P. horikoshii aminoacylase gene sequence encoded by ORF PH0722 (phoACY) can be prepared for use in the green microalgae and cyanobacteria expression constructs.

### Nucleic Acid Construct for Chloroplast Transformation.

An example of a nucleic acid construct comprising a heterologous nucleotide sequence encoding Pyrococcus horikoshii aminoacylase (that has been modified for D. salina codon usage) for chloroplast transformation of Dunaliella salina is provided by the nucleotide sequence of SEQ ID NO:3. SEQ ID NO:3 comprises a left flanking sequence of about 1000 base pairs (LB200 L-Flank: nt 1-1039), a promoter from the atpA gene (LB200 PatpA: nt 1040-1592), a first ribosome binding site (RBS1: nt 1593-1607), a nucleotide sequence conferring chloramphenicol resistance for selection (ct-cat: nt 1608-2264), a second ribosome binding site (RBS2: nt 2265-2279), a heterologous nucleotide sequence encoding Pyrococcus horikoshii aminoacylase modified for D. salina codon usage (ct-aminoacylase: nt 2280-3443), a nucleotide sequence encoding an affinity tag (His-Tag: nt 3444-3461), a terminator from the psbA gene (LB200 TpsbA: nt 3462-3849) and a right flanking sequence of about 900 base pairs (LB200 R-Flank: nt 3850-4733).

### Nucleic Acid Construct for Nuclear Transformation.

An example of a nucleic acid construct comprising a heterologous nucleotide sequence encoding Pyrococcus horikoshii aminoacylase (that has been modified for codon usage by D. salina) for nuclear transformation of Dunaliella salina is provided by the nucleotide sequence of SEQ ID NO:4. SEQ ID NO:4 comprises a promoter from the RbcS1 gene (P-RbcS1: nt 1-180), a heterologous nucleotide sequence encoding Pyrococcus horikoshii aminoacylase modified for D. salina codon usage (nu-aminoacylase: nt 181-1344), a nucleotide sequence encoding a linker (GS: nt 1345-1350), a nucleotide sequence conferring resistance to bleomycin for use in selection (ble: nt 1351-1722), a nucleotide sequence encoding an affinity tag (His-tag: nt 1723-1740), and a terminator from the RbcS1 gene (T-RbcS1: nt 1741-2076).

A further example of a nucleic acid construct for nuclear transformation of green algae is provided by the nucleotide sequence of SEQ ID NO:5. SEQ ID NO:5 is a nucleic acid construct for expressing the extromophile enzyme prolidase in Dunaliella tertiolecta UTEX LB999. SEQ ID NO:5 comprises in the following order 5′ to 3′: NotI restriction site (nt 1-8), a promoter (PrbcS1—promoter of Rubisco, small subunit 1: nt 9-188), BglII restriction site (nt 189-194), a nucleotide sequence conferring resistance to bleomycin for use in selection (ble: nt 195-569) and modified for Dunaliella nucleus codon usage, NcoI restriction site (nt 570-575), a terminator (TrbcS1—terminator of Rubisco small subunit 1: nt 576-908), NheI restriction site (nt 909-914), a promoter (PrbcS1—promoter of Rubisco, small subunit 2: nt 915-1214), KpnI restriction site (nt 1215-1220), intron 1 sequence (I1rbcS2—first intron of Rubisco small subunit 2: nt 1221-1667), SalI restriction site (nt 1668-1673), the first 771 bp of a heterologous nucleotide sequence encoding prolidase modified for Dunaliella nucleus codon usage (nt 1674-2444), intron 2 sequence (I2rbcS2—second intron of Rubisco small subunit 2: nt 2445-2697), the last 300 bp of a heterologous nucleotide sequence encoding prolidase modified for Dunaliella nucleus codon usage (nt 2698-2997), XbaI restriction site (nt 2998-3003), intron 3 sequence (I3rbcS2—third intron of Rubisco small subunit 2: nt 3004-3326), XhoI restriction site (nt 3327-3332), a terminator (TrbcS2—terminator of Rubisco small subunit 2: nt 3333-3749) and NotI restriction site (nt 3750-3757).

### Example 3

### Transformation of Green Microalgae

### Chloroplast Transformation.

Chloroplasts are transformed with the above described nucleic acid constructs using the biolistic method as known in the art and as described herein (Boynton et al. Science 240:1534-1538 (1988)). The size of gold particles and helium pressure is adjusted to obtain the highest transformation efficiencies.

Specifically, the D. salina cells are grown at about 27° C. in liquid culture comprising potassium nitrate, sodium chloride, potassium phosphate, bicarbonate and micronutrients. The D. salina cultures are grown under a light/dark regime or continuous light and supplemented with CO₂-enriched air.

### Preparation of the Microalgae Cells for Transformation:

The microalgae are concentrated by centrifugation and then resuspended in fresh media. Thus, for example, a 100 ml culture can be concentrated by centrifugation and then the pellet is resuspended in 1 mL of fresh media.

The cells are then embedded in soft agar for the bombardment procedure. For this purpose, the cells are concentrated as much as possible and a 0.8% soft agar is prepared, autoclaved, cooled to about 60° C. Chloramphenicol is added to the soft agar before mixing the agar 1:1 with the cells. The cell/agar-chloramphenicol mixture is then poured into petri dish plates and allowed to solidify. The chloramphenicol concentration in the final volume of agar and cells ranges from 200-800 mg/L.

The D. salina cells are bombarded using protocols standard in the art as described herein. The nucleic acid constructs used for transformation of the microalgae are described herein.

### Preparation of DNA for Bombardment-Binding of the DNA to the S550d Gold Carriers—5 Shots:

1. Plasmid DNA (from a stock of 1 μg/μl in TE or water) is added to 50 μl of binding buffer. Saturation can be achieved with a ratio of 4 μg selection plasmid and 10 μg of reporter plasmid per 3 mg of gold carrier.
2. The gold carrier is added to the pre-mixed plasmid DNA to insure that both plasmids bind to the carrier particles. Thus, S550d gold carrier is added to the DNA in binding buffer. Thus, for example, 60 μl (3 mg) of S550d carrier is added to the stock (plasmid in binding buffer) at 50 mg/ml. The mixture is allowed to stand for 1 minute on ice.
3. 100 μl of precipitation buffer is added to the mixture which is then allowed to stand for 1 minute.
4. The mixture in precipitation buffer is vortexed and spun (10,000 rpm in Eppendorf microfuge for 10 sec) to pellet the precipitate (i.e., the gold pellet).
5. The supernatant is removed and the gold pellet is washed with 500 μl of cold 100% ethanol. There is no need to resuspend the pellet at this step. The pellet is spun briefly in the microfuge, the supernatant removed and 50 μl of ice-cold ethanol is added.

### Transformation Using a Hand Held Gene Gun:

1. The pellet of gold beads is resuspended with a brief (1-2 sec) burst using a bath sonicator. Immediately, 5 or 10 μl of the resuspended gold beads is transferred to a grid. Aggregation is kept to a minimum by sonication immediately before applying the gold to the grid.
2. The grid is secured on the gene gun and the particles are launched (propelled) using helium at the desired pressure with the plate placed at an appropriate distance.

The bombarded cells are grown for 10-30 days at room temperature under fluorescent lights. The presence of chloramphenicol in the agar/cell mixture allows for the immediate selection of the transformed cells which develop into colonies while the untransformed cells die.

### Nuclear Transformation

Nuclear transformation of Dunaliella can be accomplished using the nucleic acid constructs as described herein and any methods for transformation as described herein and as is well known in the art.

### Example 4

### Transformation of Cyanobacteria

Cyanobacteria are transformed using the nucleic acid constructs described above for Dunaliella salina and as described in the literature (See, e.g., Frigaard et al. Methods Mol. Biol. 274:314-322 (2004)). Thus, for transformation of the cyanobacterium, Synechococcus spp., fresh cells are mixed with the nucleic acid construct. The cells are then incubated under nonselective conditions to permit DNA uptake, recombination of the inactivation construct into the genome, and expression of the antibiotic resistance marker gene. The cells are then transferred to selective conditions that only allow successful transformants to grow. In the final step, PCR is used to verify that the desired gene replacement has taken place and that the alleles have fully segregated in the isolated clone.

The detailed protocol for transformation of Synechococcus spp. is as follows.
1. Prepare an exponentially growing culture of Synechococcus sp. at 38° C. in liquid medium containing 10 mM glycerol and bubbled with sterile air supplemented with 1% CO2. Harvest the cells and resuspend them in fresh medium to an OD730 nm of 2 to 3.
2. Mix 1 to 5 μg of DNA and 0.8 mL of culture and incubate under strong illumination (about 250 μE m-2 s-1) at 38° C. for 5 h while gently bubbling with sterile air supplemented with 1% CO2.
3. Spread the cell suspension over a plate with solid medium containing 10 mM glycerol and allow the cells to grow under moderate illumination (about 150 μE m-2 s-1) at 30° C. for about 3 days until a thin lawn of growth is visible.
4. Overlay the plate with sterile, melted 0.8% (w/v) agar in water containing antibiotic (not warmer than about 50° C.) over the cells. Use about 3 mL of agar/antibiotic per plate (diameter 9 cm) containing 40 to 50 mL of solid medium.
5. Incubate the plate under the same conditions as in step 3 until single colonies appear.
6. Transfer the colonies onto fresh solid medium containing 10 mM glycerol and the appropriate antibiotic and incubate again. Re-streaking is repeated until a homogeneous isolate is obtained.

### Example 5

### Extraction and Purification of Extremozymes

The stably transformed Dunaliella salina cells are grown in liquid culture containing potassium nitrate, sodium chloride, potassium phosphate, and bicarbonate in addition to micronutrients. The culture is grown under a light/dark regime or continuous light and supplemented with CO₂-enriched air.

Harvesting and isolation of the protein and lipids is as follows. First the D. salina liquid culture is concentrated through centrifugation. The concentrated green microalgae pellet is then resuspended in about 5 volumes of water which results in the bursting of the cells and the release of the cellular contents into the water. The cell resuspension is agitated for 2 min and again centrifuged. Centrifugation separates the neutral lipid which will float to the surface from cell debris. The cell debris including chloroplasts are pelleted. The neutral lipids (triacylglycerides) can be collected for further processing for the production of biofuel.

The proteins in the pellet are resuspended and agitated in buffer that inhibits proteinases (e.g. protease inhibitor cocktail) and allows solubilization of membranes and proteins (thus, includes appropriate detergents (brij-50, tween-20 or others). The resuspended protein solution is then centrifuged. After centrifugation, the soluble supernatant contains the proteins. The proteins are separated either based on the TAG fused to the protein (see below, HIS, CBD, GST, or MBD) or via chromatography. The proteins can be further enriched by using physical treatments that correspond to the type of extremophile enzyme produced. For example, for enrichment of thermophilic/hyperthermophilic extremozymes, the protein extracts can be heat-treated at temperatures ranging from 60-100° C. (depending on the heat stability of the extremozyme) in order to denature and remove heat labile host proteins from the extracts. For halophilic extremozymes, extracts can be treated with salt solutions to salt out host protein; acidophilic and alkaliphilic extremozymes can be enriched in extracts by pH treating the extracts.

The above examples clearly illustrate the advantages of the invention. Although the present invention has been described with reference to specific details of certain embodiments thereof, it is not intended that such details should be regarded as limitations upon the scope of the invention except as and to the extent that they are included in the accompanying claims.

Throughout this application, various patents, patent publications and non-patent publications are referenced. The disclosures of these patents, patent publications and non-patent publications in their entireties are incorporated by reference herein into this application in order to more fully describe the state of the art to which this invention pertains.

1-23. (canceled)

24. A method for producing one or more extremophile enzymes, the method comprising:
(a) culturing a green microalgae cell, wherein the green microalgae cell is stably transformed with a heterologous nucleotide sequence encoding one or more extremophile enzymes and expresses the one or more extremophile enzymes; and
(b) collecting the one or more extremophile enzymes from the green microalgae cell culture of (a), thereby producing one or more extremophile enzymes.

25. The method of claim 24, wherein the green microalgae cell is stably transformed with the heterologous nucleotide sequence encoding one or more extremophile enzymes, by
propelling the heterologous nucleotide sequence at a green microalgae cell embedded in a gel at a velocity sufficient to pierce the cell wall, cell membrane and chloroplast membrane and deposit the heterologous nucleotide sequence within a chloroplast of the green microalgae cell;
wherein the heterologous nucleotide sequence is incorporated into the chloroplast genome of the green microalgae cell, thereby producing a stably transformed green microalgae cell, and
further wherein the heterologous nucleotide sequence is carried by a microprojectile and the heterologous nucleotide sequence is propelled at the green microalgae cell by propelling the microprojectile at the green microalgae cell.

26. The method of claim 24, wherein the heterologous nucleotide sequence comprises a nucleic acid construct comprising in the following order from 5′ to 3′:
(a) a left flanking sequence for homologous recombination (FS1);
(b) a first promoter (P1);
(c) a first enhancer sequence (EN1);
(d) a heterologous nucleotide sequence encoding one or more extremophile enzymes (NSEE);
(e) a first terminator (T1); and
(f) a right flanking sequence for homologous recombination (FS2);
wherein the one or more extremophile enzymes (NSEE) are modified for codon usage bias for the green microalgae cell; and
wherein FS1 and FS2 comprise nucleotide sequences that are homologous to the chloroplast genome of the green microalgae cell.

27. The method of claim 24, wherein the one or more extremophile enzymes are selected from the group consisting of an oxidoreductase, a transferase, a hydrolase, a lyase, an isomerase, a ligase, and any combination thereof.

28. The method of claim 24, wherein the green microalgae cell is a cell wall-less green microalgae cell.

29. The method of claim 24, wherein the green microalgae cell is selected from the group consisting of Dunaliellaceae, Characiochloridaceae, Chlamydomonadaceae, Golenkiniaceae, Spondylomoraceae, Tetrabaenaceae,Volvocaceae, Haematococcaceae, Asteromonadaceae, Astrephomenaceae, Phacotaceae, Oocystaceae, Chlorellaceae, Eremosphaeraceae and Characiosiphonaceae.

30. The method of claim 24, wherein the green microalgae cell is selected from the group consisting of Dunaliella salina, Dunaliella tertiolecta, Dunaliella primolecta, Dunaliella acidophilia, Dunaliella bardawil, Dunaliella lateralis, Dunaliella maritima, Dunaliella minuta, Dunaliella parva, Dunaliella peircei, Dunaliella polymorpha, Dunaliella pseudasalina, Dunaliella quartolecta, Dunaliella viridis, Dunaliella sp. SPMA, and uncultured Dunaliella.

31. A nucleic acid construct for plastid transformation of a green microalgae cell comprising in the following order from 5′ to 3′:
(a) a left flanking sequence for homologous recombination (FS1);
(b) a first promoter (P1);
(c) a first enhancer sequence (EN1);
(d) a heterologous nucleotide sequence encoding one or more extremophile enzymes (NSEE);
(e) a first terminator (T1); and
(f) a right flanking sequence for homologous recombination (FS2);
wherein the one or more extremophile enzymes (NSEE) are modified for codon usage bias for the green microalgae cell; and
wherein FS1 and FS2 comprise nucleotide sequences that are homologous to the chloroplast genome of the green microalgae cell.

32. The nucleic acid construct of claim 31, further comprising a selection cassette comprising in the following order from 5′ to 3′:
(a) a second promoter (P2);
(b) a second enhancer sequence (EN2);
(c) a nucleotide sequence for selection which confers resistance to a selection agent or encodes a selection protein (NSS); and
(d) a second terminator (T2),
wherein the NSS is modified for codon usage bias for the green microalgae cell and P2, EN2, NSS and T2 are operably located 3′ of FS1, and 5′ of P1, and
wherein the selection cassette is operably located immediately downstream of FS1 and upstream of P1 or immediately downstream of T1 and upstream of FS2.

33. The nucleic acid construct of claim 31, wherein the first and/or second promoter is selected from the group of promoters consisting of a promoter of σ⁷⁰-type plastid rRNA gene (Prrn), a promoter of the psbA gene (PpsbA), a promoter of the psaA gene (PpsaA), a promoter of the psbD gene (PpsbD), a promoter of the ATPase alpha subunit gene (PatpA), and a promoter of the RuBisCo large subunit gene (PrbcL), and any combination thereof.

34. The nucleic acid construct of claim 32, wherein the first and/or second terminator is selected from the group of terminators consisting of a terminator of the psbA gene (TpsbA), a terminator of the psaA gene (TpsaA), a terminator of the psbD gene (TpsbD), a RuBisCo large subunit terminator (TrbcL), a terminator of the σ⁷⁰-type plastid rRNA gene (Trrn), and a terminator of the ATPase alpha subunit gene (TatpA), and any combination thereof.

35. The nucleic acid construct of claim 31, wherein the one or more extremophile enzymes is an extremophile enzyme selected from the group consisting of an oxidoreductase, a transferase, a hydrolase, a lyase, an isomerase, a ligase, and any combination thereof.

36. The nucleic acid construct of claim 35, wherein the ligase is selected from the group consisting of an acyl synthetase, a carboxylase, a nucleic acid ligase, a peptide synthetase, and any combination thereof.

37. A method for stably transforming a green microalgae cell with a heterologous nucleotide sequence, the method comprising:
propelling the heterologous nucleotide sequence at a green microalgae cell embedded in a gel at a velocity sufficient to pierce the cell wall, cell membrane and chloroplast membrane and deposit the heterologous nucleotide sequence within a chloroplast of the green microalgae cell;
wherein the heterologous nucleotide sequence is incorporated into the chloroplast genome of the green microalgae cell, thereby producing a stably transformed green microalgae cell, and
further wherein the heterologous nucleotide sequence is carried by a microprojectile and the heterologous nucleotide sequence is propelled at the green microalgae cell by propelling the microprojectile at the green microalgae cell.

38. The method of claim 37, wherein the heterologous nucleotide sequence encodes one or more extremophile enzymes.

39. The method of claim 38, wherein the one or more extremophile enzymes are an oxidoreductase, a transferase, a hydrolase, a lyase, an isomerase, a ligase, or any combination thereof.

40. The method of claim 37, wherein the heterologous nucleotide sequence comprises a nucleic acid construct for plastid transformation of a green microalgae cell comprising in the following order from 5′ to 3′:
(a) a left flanking sequence for homologous recombination (FS1);
(b) a first promoter (P1);
(c) a first enhancer sequence (EN1);
(d) a heterologous nucleotide sequence encoding one or more extremophile enzymes (NSEE);
(e) a first terminator (T1); and
(f) a right flanking sequence for homologous recombination (FS2);
wherein the one or more extremophile enzymes (NSEE) are modified for codon usage bias for the green microalgae cell; and
wherein FS1 and FS2 comprise nucleotide sequences that are homologous to the chloroplast genome of the green microalgae cell.

41. The method of claim 40, wherein the heterologous nucleotide sequence further comprises one or more lipid modifying and/or lipid producing enzymes.

42. The method of claim 37, wherein the green microalgae cell is a cell wall-less green microalgae cell.

43. The method of claim 37, wherein the green microalgae cell is selected from the group consisting of Dunaliellaceae, Characiochloridaceae, Chlamydomonadaceae, Golenkiniaceae, Spondylomoraceae, Tetrabaenaceae, Volvocaceae, Haematococcaceae, Asteromonadaceae, Astrephomenaceae, Phacotaceae, Oocystaceae, Chlorellaceae, Eremosphaeraceae and Characiosiphonaceae.

44. The method of claim 37, wherein the green microalgae cell is selected from the group consisting of Dunaliella salina, Dunaliella tertiolecta, Dunaliella primolecta, Dunaliella acidophilia, Dunaliella bardawil, Dunaliella lateralis, Dunaliella maritima, Dunaliella minuta, Dunaliella parva, Dunaliella peircei, Dunaliella polymorpha, Dunaliella pseudasalina, Dunaliella quartolecta, Dunaliella viridis, Dunaliella sp. SPMA, and uncultured Dunaliella.

45. A stably transformed green microalgae cell produced by the method of claim 37.

46. A stably transformed green microalgae cell produced by the method of claim 38.

47. A method for producing lipids and extremophile enzymes in a green microalgae cell, the method comprising:
(a) culturing the transformed green microalgae cell of claim 38 that expresses one or more extremophile enzymes, wherein the green microalgae cell further produces endogenous lipids; and
(b) collecting the endogenous lipids and the one or more extremophile enzymes from the green microalgae cell culture of (a), thereby producing lipids and extremophile enzymes in a green microalgae cell.

48. A method for producing modified lipids and extremophile enzymes in a green microalgae cell, the method comprising:
(a) culturing the stably transformed green microalgae cell of claim 38 expressing one or more enzymes for modifying lipids and one or more (other) extremophile enzymes, for a time sufficient for the one or more enzymes for modifying lipids to modify the lipids produced in the green microalgae cell; and
(b) collecting the modified lipids and the one or more extremophile enzymes in the green microalgae cell culture of (a), thereby producing modified lipids and extremophile enzymes in a green microalgae cell.
